(19) United States
(12) Patent Application Publication
(10) Pub. No.: US 2021/0032674 A1
(43) Pub. Date: Feb. 4, 2021

(54) METHOD FOR DETERMINING MICROORGANISM CONCENTRATION

(71) Applicant: Q-LINEA AB, Uppsala (SE)

(72) Inventors: Jan GRAWÉ, Uppsala (SE); Markus KLINTSTEDT, Uppsala (SE)

(73) Assignee: Q-LINEA AB, Uppsala (SE)

(21) Appl. No.: 16/962,980

(22) PCT Filed: Jan. 22, 2019
(86) PCT No.: PCT/EP2019/051525

§ 371 Date: Jul. 17, 2020

(30) Foreign Application Priority Data
Jan. 22, 2018 (GB) 1801022.3

(51) Int. Cl. C12Q 1/04; C12Q 1/6806

(52) U.S. Cl. CPC C12Q 1/04; C12Q 2563/107; C12Q 1/6806

(57) ABSTRACT

The present invention provides a method of preparing a suspension of intact microorganisms from a sample containing microorganisms and mammalian cells, comprising contacting the sample with a buffer solution with a pH of at least pH 6 and less than pH 9, a detergent and one or more proteases to allow lysis of the mammalian cells; filtering the mixture through a filter suitable for retaining microorganisms to remove the lysed mammalian cells; resuspending the microorganisms retained by the filter in a liquid to provide a suspension comprising the recovered intact microorganisms; and determining the concentration of microorganisms in the suspension by: i. heating and/or contacting an aliquot of the suspension with an alcohol; ii. optionally diluting one or more aliquots of the suspension to provide one or more diluted aliquots before, during and/or after step (i); iii. contacting at least a portion of an aliquot of step (i) or (ii) with a single fluorescent stain capable of binding to DNA; iv. imaging the mixture of step (iii) at the emission wavelength of the fluorescent stain and determining an image analysis value for the number of objects corresponding to microorganisms in the imaged mixture; and v. comparing the image analysis value to a pre-determined calibration curve, thereby to determine the concentration of microorganisms in the suspension.

The present invention relates generally to the detection and characterisation of microorganisms in a sample. In particular, the present invention provides a method for recovering microorganisms from a sample containing both microbial and non-microbial cells and rapidly measuring the concentration of intact microorganisms recovered from the sample. The intact microorganisms may be viable.

Traditionally, microbial growth and the concentration of microorganisms in a sample have been determined by measuring an optical parameter of the sample, such as its turbidity. For example, McFarland standards are used in microbiology as a reference for the turbidity of a sample, so that the number of microorganisms (typically bacteria) within a sample will be within a given range of turbidity, and such standards can be used in a nephelometer to determine the concentration of microorganisms in a sample. Alternative techniques comprising spectrophotometry to determine the concentration of microorganisms in a sample may be used. However, although rapid and easily implemented, such techniques are only capable of approximating the number of microorganisms in a sample. The relationship between turbidity or the absorbance of a particular wavelength of light and the concentration of microorganisms in a sample also varies for different microorganism species, making it difficult to estimate the concentration of microorganisms when the identity of the microorganism in question is not known. Furthermore, such techniques are only capable of measuring the total turbidity or absorbance of a sample, and thus cannot distinguish between intact microorganisms or cellular or other debris in the sample. Turbidimetric measurement of the concentration of microorganisms in a sample also has low sensitivity, and a relatively high concentration of microorganisms is required in order to be able to measure the concentration of microorganisms in a sample. This prevents low concentrations being measured in this way, and may also require an extended culture step before a measurement can be made.

The number of intact microorganisms in a sample can also be estimated more quantitatively by plating a portion (or a diluted portion) of the sample on a solid growth medium, incubating the sample, and counting the number of colonies which are formed. The number of colony-forming units (CFU) within the plated sample are considered to correspond to the number of live microorganisms. However, the down-side of such techniques is that it requires a lengthy incubation step in order to allow sufficient time for microbial growth to take place. Such classical techniques are therefore useful for measuring the concentration of microorganisms in a sample at a particular point in time, but are of limited use where the concentration of intact microorganisms is required quickly, e.g. to perform a test or assay on the microorganism in that sample that requires prior knowledge of the number of microorganisms present in the sample.

It is well-known in the field of microorganism detection that viable (i.e. live) cells may also be differentiated from dead cells, and a number of techniques are available for this purpose. Methods known in the art focus on nucleic acid stains, membrane potential, redox indicators or reporter genes. Typically, these techniques rely on the fact that the membrane of a viable microorganism is intact, whilst that of a dead microorganism is disrupted and/or broken (Gregori et al. 2001. Appl. Environ. Microbiol. 67, 4662-4670).

A particular technique which allows dead cells to be differentiated from live cells is live/dead staining. By using a dye or stain which is non-membrane permeable, only cells which have a disrupted membrane are stained, whilst cells which have an intact membrane are not. The dye/stain thus acts as a marker for dead cells, as only those cells with a disrupted membrane (i.e. dead cells) are stained using such a dye. In this way, dead cells may be detected, and furthermore the proportion of the total cells which are dead can be calculated. Further advances in this field have led to the development of techniques using two separate stains: a first, which is cell-permeable and can enter both live and dead cells; and a second, which is cell-impermeable, and can only enter dead cells. Live and dead cells can therefore be differentially-labelled, and may thus be distinguished. An example of a kit for performing this technique is the LIVE/DEAD BacLight Bacterial Viability Kit (Invitrogen), which comprises the SYTO9 (cell permeable) stain and propidium iodide (PI) (cell impermeable) fluorescent dyes. By detecting microbial cells at the emission wavelengths of both first and second stains used in such kits, such techniques may be particularly useful in differentiating between live and dead microbial cells, thereby to determine the proportion of viable microorganisms present in a sample.

A number of different detection techniques can be used to distinguish differentially stained live and dead microbial cells in a sample. For example, it is possible to directly count the number of microbial cells in a sample which are indicated as viable and non-viable, e.g. in a microscope field of view, and in this way determine the proportion of viable microorganisms present in a sample. However, such techniques are labour- and time-intensive, and do not allow the concentration of viable microorganisms to be accurately determined. Automated cell counting methods such as flow cytometry may also be used to measure the proportion of viable microorganisms in a sample when combined with live/dead staining techniques (Berney et al. 2007. Applied and Environmental Microbiology 73, 3283-3290). However, complex and highly specialist instrumentation, and regular calibration (e.g. a separate calibration before measuring each sample) is required in flow cytometric methods. Such techniques therefore are typically not suitable for use in a robust detection method such as is required for routine clinical laboratory use, and automation of such techniques may be difficult. There is therefore a need for straightforward, rapid and robust methods and instruments for measuring the concentration of intact microorganisms in a sample, particularly for clinical use.

As discussed above, microorganisms which have an intact cell membrane can be stained differently to those which have a damaged or disrupted cell membrane when suitable dyes are used. Detecting viable cells by live/dead staining therefore typically comprises detecting cells having an intact cell membrane, and cells having an intact cell membrane are therefore considered to represent viable cells for the purposes of measuring the concentration of viable microbial cells in a sample. The correlation between a cell being intact and being viable is good, and detecting intact cells is considered to be an effective way for the amount or concentration of viable cells in a sample to be determined.

Our co-pending application PCT/EP2018/077852 relates to a method which uses “live/dead” stains and imaging to determine the concentration of intact microbial cells in a sample. By combining aspects of such a method with a particular, gentle, way of recovering microbial cells from a sample, and pre-treating the recovered cells, the inventors of the present application have invented a method for determining the concentration of intact microbial cells recovered from a sample utilising only a single dye or stain, which provides an improved and simplified technique.

As noted above, in many instances in microbiology it is desirable to determine the concentration of microorganisms, and in particular intact microorganisms. This may be desirable to allow a suitable concentration or number of microorganisms to be provided for an assay to characterise a microorganism, so that said assay may be performed correctly, or indeed to ensure that a sample is suitable for use in a particular assay. Notably, this may include the preparation of standard (or standardised) cultures, or inocula for cultures. This includes particularly the preparation of standardised inocula for antibiotic susceptibility tests (ASTs), which for clinical purposes in the detection and identification of microbial infections require an inoculum which is of a known or predetermined, or standard, concentration. However, it may also be desirable to determine the concentration of microorganisms in a sample, or provide a standard culture, for other assays, as discussed in greater detail below.

Numerous processes in biology and medicine require the accurate determination of the number of microorganisms (particularly intact/viable microorganisms) in a sample, and the preparation of an inoculum based on said determination. These include, for example, water and food quality control analysis, monitoring of microorganisms in an environmental sample, biofilm formation in or on medical equipment or in a patient, and laboratory microbiological research. In particular, the accurate determination of the concentration of viable microbial cells in a sample, and the preparation of an inoculum containing a desired concentration of microorganisms therefrom may be of use in the diagnosis of a microbial infection.

Microbial infections represent a major class of human and animal disease with significant clinical and economic implications. Whilst various classes and types of antimicrobial agents are available to treat and/or prevent microbial infections, antimicrobial resistance is a large and growing problem in modern medicine. In the context of treatment of a microbial infection, it can therefore be desirable, and indeed important, to have information regarding the nature of the infecting microorganism and its antimicrobial susceptibility profile in order both to ensure effective treatment and also to reduce the use of unnecessary or ineffective antibiotics and thereby to help control the spread of antibiotic, or more generally antimicrobial, resistance. This is particularly so in the case of serious or life-threatening infections in which rapid effective treatment is vital. Sepsis, a potentially fatal whole-body inflammation caused by severe infection is the most expensive condition and driver of hospital costs in the US, comprising 5% of the total national hospital cost. Mortality increases 7% for every hour for severe sepsis, if not treated properly, and the rising prevalence of antimicrobial-resistant sepsis-causing strains, particularly bacterial strains, makes predictions of the correct treatment for sepsis increasingly difficult. The current gold standard for diagnosis of the microorganisms causing sepsis or other infections is based on phenotypic and biochemical identification techniques which require the isolation and culture of pure cultures of the infecting microorganisms. It can take several days to perform the microbial identification (ID) and antibiotic susceptibility (AST) tests to identify the infection and determine the susceptibility profile of the microorganism, which may be resistant to one or more antibiotics. An AST assay provides a ‘minimum inhibitory concentration’ or WIC′ value for each antimicrobial agent tested on a microorganism, and can thus provide information on which antimicrobial agents may be effective against the microorganism. The more quickly such information can be provided the better, and hence rapid AST methods are desirable and are being developed.

Generally speaking, results obtained for AST determinations in the clinical field should be comparable between different methods and/or different clinical laboratories. To this end it is customary to use prescribed and recognised conditions for AST testing. This may involve the use of prescribed medium (e.g. Muller-Hinton (MH) media) and culture conditions. In particular, it is also customary to use standardised microbial titres (i.e. a standardised (or standard) number or amount (e.g. concentration) of microbial cells) to set up the cultures which are performed (i.e. monitored for growth) in an AST test, such that the number or amount of bacteria in the cultures is at a set value. For example, McFarland standards are conventionally used as a reference to adjust the turbidity of microbial suspensions (especially bacterial suspensions) so that the number of microorganisms in the culture preparation used to set up the cultures will be within a given range to standardise AST testing. McFarland standards are set based on the turbidity of reference suspensions, and microbial suspensions are adjusted in concentration (or number of bacteria) to match the turbidity of a selected McFarland standard.

Conventionally (e.g. as described for the EUCAST standard method for determining MICs of antimicrobial agents (European Committee for Antimicrobial Susceptibility Testing (EUCAST) of the European Society of Clinical Microbiology and Infectious Diseases (ESCMID), Clinical Microbiology and Infection, Vol. 9(8): ix-xv, 2003)), microbial cells for AST (e.g. from a clinical sample culture) are plated and incubated to obtain isolated colonies. Colonies may then be collected and used to prepare a microbial cell suspension for use as the inoculum for use in the AST assay. Typically, and as described in the guidelines described above, the concentration of microorganisms in the suspension thus prepared is set to a standard and pre-defined level, e.g. 0.5 McFarland units, to allow a standard concentration of microorganisms to be used in an AST assay. The turbidity of the microbial suspension may be adjusted to 0.5 McFarland units before use. Alternatively, the isolated individual colonies may be used to inoculate a culture medium which may be cultured to provide the inoculum. The culture may be allowed to grow to the desired (0.5 McFarland unit) standard and/or may be adjusted if necessary to this standard, before it is used as the inoculum. Thus before normalising the concentration of bacteria before an AST, microbial cultures are typically allowed to grow until the growth reaches a turbidity equal to or greater than that of a 0.5 McFarland standard. If needed, the culture may be adjusted to give culture having a turbidity equivalent to the 0.5 McFarland standard. This may then be used as the inoculum that is used to set up an AST assay. The inoculum obtained at this point (i.e. the culture or suspension of approximately 0.5 McFarland units) is diluted in broth to give the desired standardised final cell number concentration used for an AST culture. By way of reference, a microbial culture/suspension of 0.5 McFarland units comprises a microbial concentration of approximately 1×10⁸ CFU/ml. Such a microbial culture/suspension would typically be diluted in broth by a factor of ˜200 when setting up an AST culture, i.e. each AST culture condition would typically comprise a starting microbial concentration of approximately 5×10⁵ CFU/ml.

For certain microbial infections, such as sepsis, a blood sample is typically collected in a blood culture flask, and a microbial culture (i.e. a clinical sample culture) is allowed to grow until a positive culture result is obtained in a culture monitoring system. In automatic culture detection systems such as e.g. Bactec or Bact/Alert systems the concentration of bacteria needed to be indicated as positive is between 10⁸ to 10⁹ CFU/ml, corresponding to 0.5 to 3.5 McFarland units (if measured in a saline solution). The lowest McFarland value that is readily detectable (either by eye or by turbidimetric measurements) is around 0.5 McFarland units.

ID tests and AST determination may be performed using such a clinical sample culture, generally once a positive culture result has been obtained. For an AST test, it is typical to prepare a further culture from the clinical sample culture (e.g. a positive culture) to use as, or for preparing, an inoculum for the AST test cultures and to standardise such an inoculum to a pre-set microbial concentration or McFarland value (typically 0.5 McFarland units) before it is used to inoculate the AST test cultures. Thus inocula for AST are typically prepared using, or starting from, cultures or microbial suspensions which are at 0.5 McFarland units. This is typically done in the methods of the art by selecting colonies obtained by plating the clinical sample culture or microorganisms isolated therefrom as described above.

Techniques which require comparison with McFarland standards to determine the concentration of microorganism in a sample only provide an approximation for the concentration, and fail to provide information specifically on the concentration of viable microbial cells in a sample. Furthermore, such techniques rely on the concentration of microorganisms in the sample being relatively high (e.g. 0.5 McFarland units) in order for the concentration to be measured.

There is therefore a particular need to improve the speed and sensitivity with which the concentration of microorganisms in a sample is determined, particularly in the context of setting up an AST assay. In particular, there is a need for a robust and simple method which allows the rapid, accurate and sensitive microorganism concentration determination to be performed without requiring complex instrumentation, such as methods which comprise flow cytometry. The present invention addresses this need by providing an improved method for determining the concentration of a microorganism, which may be used in the preparation of a microbial inoculum, and further to provide an improved workflow for performing an AST, and which allows the concentration of microorganisms in a microbial suspension, and more significantly the concentration of intact microorganisms in a microbial suspension, to be accurately and rapidly determined. In particular, the concentration determination method of the present invention is of value in enabling a rapid AST assay to be performed. Thus, the present invention provides a rapid, accurate and precise method for determining the concentration of microorganism in a microbial preparation, and more significantly the concentration of intact microorganisms. As noted above, the concentration of intact microorganisms may be used as a reliable indicator of viable microorganisms.

In particular, the method of the present invention is based on recovering microbial cells from a sample in a way which is particularly effective at separating microbial cells from non-microbial (particularly mammalian) cells, by lysing the non-microbial cells whilst leaving the microbial cells intact (and largely or essentially viable), staining intact microorganisms in a suspension of the recovered microorganisms, and imaging the suspension in order to determine a value for the number of objects corresponding to intact microorganisms in the sample, rather than directly counting microorganisms or estimating the concentration of microorganisms turbidimetrically with respect to a pre-determined standard, or counting the number of viable microorganisms present by counting cultured colonies. By using a predetermined standard curve, the determined values for the number of objects detected by imaging may be correlated to the concentration of microorganisms present in the suspension. By combining such a gentle (viability-preserving) separation (microbial isolation) technique with a step of pre-treating the recovered microbial cells with an alcohol and/or heat prior to staining, to assist in or aid the staining process, it has surprisingly been found only a single stain may reliably be used in order to detect and determine the concentration of microorganisms in the suspension of recovered microorganisms. Without wishing to be bound by theory, we believe that that the isolation step yields sufficiently pure microbial cell preparations from the sample which are largely (e.g. substantially or essentially) viable (e.g. with only a small proportion of non-viable microbial cells), which allows the use of only a single stain. This combined effect, together with the pre-treatment, is especially beneficial for quantitating microorganisms, particularly bacteria, with resistance mechanisms that affect cell permeability, and hence ability of the microorganism to take up and/or retain a stain. Without wishing to be bound by theory, it is believed that the pre-treatment step may disrupt or disable efflux pumps (a common antimicrobial resistance mechanism) in microorganisms, thereby enhancing the staining of resistant microorganisms in particular, or indeed any microorganism with a strong or effective efflux pump. In other words, staining of microorganisms (particularly antimicrobial resistant microorganisms) may be normalised by the use of a pre-treatment step described herein. This is important as mistakes in AST for resistant bacteria are especially harmful for the patient from whom the bacteria have been isolated as it leads to increased risk for wrong treatment.

Accordingly, in a first aspect, the present invention provides a method of preparing a suspension of intact microorganisms from a sample containing microorganisms and mammalian cells, said method comprising:
a. providing a sample containing microorganisms and mammalian cells;
b. contacting said sample with a buffer solution, a detergent and one or more proteases, wherein said buffer solution has a pH of at least pH 6 and less than pH 9 to allow lysis of mammalian cells present in said sample;
c. filtering the mixture obtained in step (b) through a filter suitable for retaining intact microorganisms, wherein said filtering removes the lysed mammalian cells from the mixture;
d. recovering the microorganisms retained by the filter in step (c), wherein said recovery comprises re-suspending the microorganisms in a liquid to provide a suspension comprising the recovered intact microorganisms; and
e. determining the concentration of microorganisms in said suspension, wherein the concentration of microorganisms is determined by a method comprising:
   i. contacting an aliquot of said suspension with an alcohol and/or heating an aliquot of said suspension;
   ii. optionally diluting one or more aliquots of said suspension to provide one or more diluted aliquots at one or more dilution values, wherein said dilution takes place before, during and/or after step (i);
   iii. contacting at least a portion of an aliquot of step (e)(i) or (e)(ii) with a single fluorescent stain capable of binding to DNA to provide a suspension-stain mixture, wherein said stain has an emission wavelength;
   iv. imaging the suspension-stain mixture of step (e)(iii) at the emission wavelength of the fluorescent stain and determining an image analysis value for the number of objects corresponding to microorganisms in the imaged mixture; and
   v. comparing an image analysis value obtained in step (e)(iv) for a said aliquot of step (e)(iii) to a pre-determined calibration curve, thereby to determine the concentration of microorganisms in the suspension.

It will be understood from the above that step (b) is a step of selective lysis of non-microbial cells present in the sample, which leaves microbial cells in the sample intact (or more particularly substantially intact). Thus, in step (b) the detergent is used in an amount or concentration which is effective to lyse (or which acts to lyse, or is capable of lysing) non-microbial cells, but which is not effective to lyse (or which does not act to lyse, or is not capable of lysing) microbial cells.

As noted above, step (e)(i) of pre-treating the microorganisms in the suspension with alcohol and/or heat acts to facilitate the subsequent staining. Without wishing to be bound by theory, this may be due, at least in part, to an effect of the pre-treatment in permeabilising the cell wall and/or membrane of the microorganisms, or otherwise effecting conformational changes in the structure of the microorganism, to facilitate entry and/or retention of the stain, and/or in inactivating the microorganisms, for example so that the stain is not removed from the microbial cell by an efflux pump. As noted above, the inactivation of an efflux pump in microorganisms where they are present is believed to be an important contributor to the beneficial effects of the method. Thus, alternatively expressed, in step (e)(i) the pre-treatment may act to normalise the staining.

Whilst alcohol and/or heat provide an effective such pre-treatment, this may also be achieved by other means, for example the use of detergents, e.g. at concentrations or in amounts which are able to achieve a similar (e.g. permeabilising and/or inactivating) effect on the microorganisms.

Accordingly, in another aspect, the invention provides a method of preparing a suspension of intact microorganisms from a sample containing microorganisms and mammalian cells, said method comprising:
a. providing a sample containing microorganisms and mammalian cells;
b. contacting said sample with a buffer solution, a detergent and one or more proteases, wherein said buffer solution has a pH of at least pH 6 and less than pH 9 to allow lysis of mammalian cells present in said sample;
c. filtering the mixture obtained in step (b) through a filter suitable for retaining microorganisms, wherein said filtering removes the lysed mammalian cells from the mixture;
d. recovering the microorganisms retained by the filter in step (c), wherein said recovery comprises resuspending the microorganisms in a liquid to provide a suspension comprising the recovered intact microorganisms; and
e. determining the concentration of microorganisms in said suspension, wherein the concentration of microorganisms is determined by a method comprising:
   i. contacting an aliquot of said suspension with a detergent;
   ii. optionally diluting one or more aliquots of said suspension to provide one or more diluted aliquots at one or more dilution values, wherein said dilution takes place before, during and/or after step (i);
   iii. contacting at least a portion of an aliquot of step (e)(i) or (e)(ii) with a single fluorescent stain capable of binding to DNA to provide a suspension-stain mixture, wherein said stain has an emission wavelength;
   iv. imaging the suspension-stain mixture of step (e)(iii) at the emission wavelength of the fluorescent stain and determining an image analysis value for the number of objects corresponding to microorganisms in the imaged mixture; and
   v. comparing an image analysis value obtained in step (e)(iv) for a said aliquot of step (e)(iii) to a pre-determined calibration curve, thereby to determine the concentration of microorganisms in the suspension.

In particular, in step (b) the detergent is effective to (or acts to) achieve (or is capable of achieving) a selective lysis of non-microbial cells (i.e. to lyse non-microbial cells in the sample, but not to lyse microbial cells), whereas in step (e)(i) the detergent is effective to (or acts to) facilitate (or is capable of facilitating), e.g. to enhance or improve or allow or normalise, staining of microbial cells, particularly antimicrobial resistant microbial cells or microorganisms with strong efflux pumps. Thus, whilst the same or different detergents may be used in steps (b) and (e)(i), where the detergent is the same, it will be used in a different (higher) amount in step (e)(i) compared to step (b).

In the above-mentioned methods the fluorescent stain may be cell-permeable or cell-impermeable, but in a preferred embodiment it is cell-permeable.

Whilst the pre-treatment step may affect the permeability of the cell membrane and/or cell wall of the microorganism, and hence may have an effect on the integrity of the cell wall and/or membrane, we have found that this does not detract from being able to detect and image the microorganisms for enumeration of objects corresponding to microorganisms. Thus, objects corresponding to microorganisms may be identified and may be imaged. Although in the pre-treatment step the cell wall and/or membrane integrity may be disrupted to some degree, the imaged objects can be identified as corresponding to microorganisms which were recovered as intact in step (d). Accordingly, the image analysis value which is obtained in step (e)(iv) may be seen as a value for the number of objects in the imaged mixture corresponding to (or representative of) recovered intact microorganisms. Thus, in step (e)(v) the comparison step allows the determination of the concentration of intact microorganisms in the suspension (i.e. in the suspension prepared in step (d)).

The comparison in step (e)(v) of the image analysis value for the number of objects detected with a pre-determined calibration curve enables a more accurate measure of the number of microorganisms (or more particularly intact microorganisms) in the suspension to be obtained. Various factors can affect the staining and or determination of intact cells by staining methods. For example, in the context of live/dead staining, it has in some cases been reported that whilst a proportion of microbial cells which are indicated as ‘viable’ in a live/dead staining assay may comprise an intact cell membrane, they may, as a matter of fact, be metabolically inactive or otherwise non-culturable (Trevors 2012. J Microbiol Meth 90, 25-8). Furthermore, during fast exponential growth in nutrient rich environments the membrane integrity of viable microbial cells may be reduced, thereby allowing the second fluorescent dye to enter the cells (Shi et al. 2007. Cytom Part A 71A, 592-298). Such cells would therefore emit light at the second emission wavelength, and due to the ability of the second fluorescent stain to quench the fluorescence of the first fluorescent stain, the fluorescence of such cells at the first emission wavelength may be reduced. Additionally, problems such as bleaching and higher than expected uptake of the first (cell permeable) fluorescent stain may affect the accuracy of such methods (Stiefel et al. 2015. BMC Microbiology 15:36). The methods disclosed herein allow such factors which can adversely affect the determination of the concentration of intact cells in a sample to be taken into account (i.e. ‘factored in’ to any such determination), thereby resulting in a more accurate measure of microbial viability. Thus, the concentration which is determined for intact microbial cells may be taken to represent, or to indicate or correspond to, or approximate, the concentration of viable microbial cells. Specifically, by comparing the image analysis value for the number of objects imaged in step (e)(iv) with a pre-determined calibration curve, factors such as incorrect staining of viable and non-viable microbial cells discussed above can be taken into account when attempting to calculate the concentration of intact, and more particularly viable, microorganisms present in a suspension, thus allowing a more accurate determination of the concentration of intact or viable microorganisms in a suspension to be made.

The present invention provides a rapid and sensitive method for determining the concentration of microorganisms in a suspension prepared from a sample (or, alternatively expressed in a sample of recovered microorganisms, a “recovered microorganism sample”). This may have a number of utilities and it can be advantageous to have a robust and simple method for determination of microbial concentration in recovered microorganism samples a number of situations. As well as accurately determining absolute concentrations of microorganisms, the method may also have utility in giving an indication of microbial load in a sample, and thus may be of use in any method or context where it is desired to know or to estimate, or have an idea of, how many microbial cells are present. The context in which this method may be used is therefore not limited. Indeed, given the low limit of detection of this method, this method may be used to determine whether or not a sample contains microorganisms. Thus, in one aspect the present invention provides a method for determining the presence of a microorganism in a sample, said method comprising performing steps (a)-(e) of either of the above methods disclosed herein, and determining whether microorganisms are present in the sample.

The methods of the invention may have utility in the context of different samples or suspensions where it may be desirable to assess or determine microbial concentration. The sample contains both microorganisms and mammalian cells, and thus is preferably derived from a mammal. The sample may in particular be a clinical sample or veterinary sample, as discussed further below. The methods may be used to determine if a sufficient or appropriate concentration of cells is recovered from the sample to enable further tests to be carried out. This is described further below in the context of an AST assay, but the method may be used as a preliminary step before any step of subsequent analysis of the microorganisms in the sample. For example the method may be used to determine or assess the concentration of intact (or viable) microorganisms in a sample before carrying out mass-spectroscopy tests, and/or nucleic acid based tests, and/or any other evaluation of the microorganism, e.g. growth-based studies.

Once the concentration of intact (or viable) microorganisms in a suspension of recovered microorganisms has been determined, this information may advantageously be used to accurately prepare an inoculum containing a known or desired number or concentration of microorganisms.

Accordingly, in a further aspect the invention provides a method of preparing a microbial inoculum (or, alternatively expressed, an inoculum for use in preparing a microbial culture), said method comprising recovering and determining the concentration of microorganisms in a suspension using a method defined herein, and then adjusting the concentration of microbial cells in at least an aliquot or portion of the suspension to a desired concentration, thereby to provide an inoculum comprising a desired concentration of microorganisms.

The present invention also provides methods for characterising a microorganism in a sample once the concentration of microorganisms in a suspension comprising microorganisms recovered from said sample has been determined. Thus, the recovery and concentration determination method of the present invention may be used in conjunction with an assay for characterising a microorganism. In particular, this may be an assay which requires a known or pre-determined concentration or number of microorganisms.

Thus, in another aspect, the present invention provides a method for characterising a microorganism in a sample, said method comprising:

(i) providing a sample containing microorganisms and mammalian cells;

(ii) performing steps (b)-(d) as defined above on said sample, to yield a suspension of the intact (e.g. viable) microorganisms;

(iii) performing step (e) as defined above to determine the concentration of microbial cells in the suspension;

(iv) adjusting the concentration of microbial cells in said suspension, if necessary, to a desired or pre-determined concentration; and

(v) characterising the microorganism in the suspension (and hence in the sample).

The present invention therefore allows the concentration of microorganisms in a preparation (suspension) of recovered microorganisms to be determined prior to performing an assay, particularly an assay which requires a particular concentration or number of microorganisms, to characterise said microorganism. This therefore allows it to be determined whether a sample, or more particularly a suspension prepared therefrom, is suitable for use in a given assay, and if not, allows the concentration of microorganisms to be adjusted appropriately.

Whilst a concentration adjustment step in any of the methods set out herein may beneficially be informed by the concentration determined for the microorganisms in the suspension, it is not required that all steps of the concentration adjustment take place after the concentration determination has been completed (e.g. after step (iii) in the method above). In an embodiment the adjustment may take place after the concentration has been determined, for example one or more dilution steps are performed after the concentration has been determined. However, in other embodiments, an initial (i.e. preliminary) adjustment step may take place before the step of concentration determination is completed, or separately, e.g. whilst the concentration determination is being performed, or before. For example, a preliminary dilution step of the suspension or a part thereof may take place before the concentration has been determined. (This is separate from the optional dilution step of the aliquot in step (e)(ii) in the concentration determination method). In such an embodiment, one or more further dilution steps may then take place after the concentration has been determined, in order to arrive at a desired concentration (i.e. the dilution resulting from such an initial (preliminary) dilution may be further diluted). Such a further dilution is informed by (or based on) the determined concentration. It will be understood in this respect that such an initial (or preliminary) dilution step (which may be viewed as a “blind” dilution step) will take place on a portion of the suspension which is different from the aliquot of the suspension on which the concentration determination is performed. Thus, the remainder of the suspension (that is the suspension remaining after the aliquot has been removed for concentration determination) may be adjusted (e.g. diluted) in a preliminary adjustment step, or a separate portion or aliquot of the suspension (i.e. remaining suspension) may be subjected to a preliminary adjustment step. This may speed up the overall method.

In a further aspect the present invention provides a method for determining the antimicrobial susceptibility of a microorganism in a sample, said method comprising:
(i) providing a sample containing a viable microorganism and mammalian cells;
(ii) performing steps (b)-(d) as defined above on said sample, to yield a suspension of the viable microorganisms;
(iii) performing step (e) as defined above to determine the concentration of microbial cells in the suspension;
(iv) inoculating a series of test microbial cultures for an antibiotic susceptibility test (AST) using the suspension of step (ii), wherein the series of test microbial cultures comprises at least two different growth conditions, wherein the different growth conditions comprise one or more different antimicrobial agents, and each antimicrobial agent is tested at two or more different concentrations; and
(v) assessing the degree of microbial growth in each growth condition;

wherein the concentration of microbial cells in said suspension or said test microbial cultures is adjusted if necessary to a desired or pre-determined concentration; and wherein the degree of microbial growth in each growth condition is used to determine at least one value indicative of the susceptibility of the microorganism in the sample to at least one antimicrobial agent.

In an embodiment, at least one MIC and/or SIR value may be determined, thereby to determine the antimicrobial susceptibility of said microorganism in said sample.

SIR is well known and understood in the art to mean sensitive, intermediate or resistant. Whilst SIR is a more course scale than MIC it is used clinically in many instances.

The present invention therefore provides a more accurate method for performing an AST assay, as it allows the concentration of microorganisms to be determined with greater accuracy than measuring turbidity of a sample (e.g. by a simple comparison of the turbidity of a sample with that of a McFarland standard). The method is also simpler than a method employing two “live/dead” stains, since only a single stain is used. A further advantage of the present methods lies in being able to determine the concentration of resistant microorganisms, and in one embodiment the microorganism is a resistant microorganism, particularly resistant bacteria. As noted above, resistance mechanisms in microorganisms, particularly bacteria, to antimicrobial agents may include a more resistant cell wall and/or membrane, and/or an efflux pump which removes the antimicrobial agent from the microbial cell. Such mechanisms may also act to impede the uptake and/or retention of a stain by the microorganism. It is believed that the methods of the invention, including particularly the pre-treatment step, may facilitate (or enhance) the staining process (particularly antimicrobial resistant microbial cells) to allow such resistant microorganisms to be detected, or measured. Put another way, the methods of the present invention, particularly the pre-treatment step, may normalise microbial staining. We have compared resistant and non-resistant bacteria, and bacteria of different types, with or without pre-treatment and have observed an enhanced similarity in staining between the different bacteria (i.e. normalised staining). Accordingly, the staining, and the methods of the invention may be performed without knowing the identity of the microorganisms.

The concentration determination steps of the above-disclosed methods may therefore have utility in determining the concentration of resistant microorganisms, particularly resistant bacteria, and may further have a more general applicability of determining the concentration of a microorganism in any suspension or preparation of a microorganism.

Accordingly, also disclosed herein is a method for determining the concentration of intact microorganisms in a suspension of microorganisms, said method comprising:
(i) providing a suspension containing microorganisms;
(ii) contacting an aliquot of said suspension with an alcohol and/or detergent and/or heating an aliquot of said suspension;
(iii) optionally diluting one or more aliquots of said suspension to provide one or more diluted aliquots at one or more dilution values, wherein said dilution takes place before, during and/or after step (ii);
(iv) contacting at least a portion of an aliquot of step (ii) or (iii) with a single fluorescent stain capable of binding to DNA to provide a suspension-stain mixture, wherein said stain has an emission wavelength;
(v) imaging the suspension-stain mixture of step (iv) at the emission wavelength of the fluorescent stain and determining an image analysis value for the number of objects corresponding to microorganisms in the imaged mixture; and
(vi) comparing an image analysis value obtained in step (v) for a said aliquot of step (iv) to a pre-determined calibration curve, thereby to determine the concentration of microorganisms in the suspension.

As for the methods above, the image analysis value determined in step (v) may be for the number of objects in the imaged mixture corresponding to intact microorganisms, and in step (vi) the concentration of intact microorganisms in the suspension may thereby be determined.

Further, as noted above, in an embodiment of this method, the microorganisms may be resistant microorganisms, more particularly resistant bacteria. Still further, such a method may be used in the context of an AST determination and so the method may be used as part of a method for a method for determining the antimicrobial susceptibility of a microorganism in a sample, analogously to that described above.

As described above, a standard AST assay performed according to EUCAST or CLSI guidelines typically requires periods of time for microorganisms to grow sufficiently to be used in the next step of setting up the AST assay. For example, in the protocol outlined above a period of incubation is required to allow the concentration of microorganisms in the clinical sample culture to increase to a point where the clinical sample culture is regarded as ‘positive’ (i.e. it reaches at least 0.5 McFarland units). Further incubation steps are required following plating of the clinical sample culture to allow individual colonies to grow, and optionally an additional further incubation step is required to allow a microbial suspension prepared as outlined above to reach 0.5 McFarland units before an AST assay can be prepared.

Furthermore, in the protocol outlined above for preparing an AST assay, typically only one or a small number of colonies (relative to the total number of microorganisms present in a clinical sample culture) are used to prepare an inoculum that is eventually used to set up an AST assay. Such a protocol therefore relies on the colony or colonies used being representative of the microorganisms causative for an infection. Where this is not the case, the results of the AST assay may not truly reflect the antimicrobial susceptibility of the microorganisms causative for an infection, and any clinical intervention based on such results may therefore fail to adequately treat the infection.

More broadly, the present invention provides methods for rapidly and accurately determining the concentration of intact microorganisms in a suspension recovered from a sample in order to allow a suitable concentration or number of microbial cells to be used in a qualitative or quantitative assay to characterise said microorganism. Put another way, the concentration of intact microorganisms in a recovered suspension may be determined prior to any desirable method of characterising a microorganism, in order to allow a suitable concentration or number of microbial cells to be provided for a characterisation method. This therefore allows the characterisation of a microorganism using any such assay.

Assays for which it may be particularly advantageous to determine the concentration of intact microorganisms in a suspension of microorganisms recovered from a sample include, for example, mass spectrometry (including MALDI-TOF, ESI-MS and CyTOF), Raman spectroscopy, nucleic acid-based tests (including PCR, rolling circle amplification (RCA), ligase chain reaction (LCR), and nucleic acid sequence based amplification (NASBA), which may be of particular utility in identifying a microorganism and/or a marker for antimicrobial resistance therein). As described in greater detail elsewhere herein, it may be of particular benefit to determine the concentration of intact microorganisms in suspension prepared from a sample prior to performing an AST assay.

As used herein, the terms “microbial cell” and “microorganism” are interchangeable and are considered to have equivalent meanings, namely a microscopic organism. The term is used broadly herein to include all categories of microorganism, whether unicellular or not, and includes bacteria, including mycobacteria, archaea, fungi, protists, including protozoa, and algae, as discussed in greater detail below. The identity of the microorganisms may be known or unknown when the method is carried out. Further the sample may contain one type or species of microorganism or more than one type or species, i.e. the sample may contain a single type of microorganism or may contain a mixture of microorganisms.

Furthermore, reference to “cell-permeable” and “cell-impermeable” stains is made in reference to microbial cells. In other words, the permeability of the stains used in the methods of the present invention is the permeability of microorganisms to said stains.

The term “viable” in the context of the present invention refers to microorganisms that are able to grow and/or reproduce. The concentration of viable microorganisms in a sample may be determined indirectly, by determining the concentration of intact microorganisms in the sample by differential staining. The concentration of viable microorganisms is therefore derived from the concentration of intact cells in the sample. The method of the invention provides an accurate and rapid way for determining the concentration of intact microorganisms in the sample. When a sample containing viable microorganisms is used in step (a), the determination of the concentration of intact microorganisms according to the invention reflects, or provides an indication of the concentration, of viable microorganisms.

The term “intact” in the context of the microorganisms which are present in the sample and which are recovered from the sample and present in the suspension which is prepared refers to microorganisms with no substantial change to their integrity. Such “intact” microorganisms will generally have non-disrupted cell membranes, i.e. cell membranes which are semi-permeable and retain a membrane potential (i.e. have a protein gradient). However, as noted above, the pre-treatment with alcohol or heat (or detergent) may have a permeabilising effect, and hence following the pre-treatment the microorganisms may not be intact in the strict sense of the definition above. Nonetheless, such pre-treated microorganisms are representative of intact microorganisms present in the suspension and the determination of their concentration in the pre-treated aliquot (of step (e)(i)) is therefore indicative of the concentration of intact microorganisms in the suspension. Further, the permeabilising effect of the pre-treatment, if any, may be relatively mild and insufficient fully to disrupt the microbial cells.

As detailed above, the invention provides methods of preparing a suspension of intact microorganisms. The term “suspension” is used herein with its common meaning known in the art, i.e. a mixture containing particles. In the current instance the “particles” are microorganisms and the suspension of microorganisms in the methods herein is simply a preparation comprising microorganisms in a liquid. As detailed, the suspension is prepared from a sample containing microorganisms and mammalian cells.

A range of samples containing a range of possible microorganisms may be analysed in the methods of the present invention. As stated above, the sample contains microorganisms and mammalian cells. However, it must be understood that it may not be possible to determine whether a sample of interest contains microorganisms until a method of the invention has been performed. The sample is preferably isolated from a mammal, but this not essential and the sample may be derived from elsewhere, e.g. it may be an environmental sample. The sample may be known to contain mammalian cells (e.g. if it is derived from a mammal), or may merely be suspected to contain mammalian cells, or it may be thought possible that the sample contains mammalian cells. Accordingly, as defined herein the “sample containing microorganisms and mammalian cells” may be a sample suspected to contain microorganisms and mammalian cells.

The microorganism may be any microorganism (e.g. any bacterial or fungal microorganism, or protozoa, in particular any pathogenic microorganism or any microorganism causing an infection in the body, and thus a method of the invention may in particular be used to determine the concentration of microorganisms in the context of detecting or diagnosing a microbial infection in or on any part of the body of a test subject (i.e. any microbial infection). Generally speaking, the invention is concerned with the analysis of samples containing (or suspected of containing) clinically-relevant microorganisms, but the microorganism may be pathogenic or non-pathogenic.

As used herein, the term microorganism encompasses any organism which may fall under the category of “microorganism”. Although not necessarily so, microorganisms may be unicellular, or may have a unicellular life stage. The microorganism may be prokaryotic or eukaryotic and generally will include bacteria, archaea, fungi, algae and protists, including notably protozoa. Of particular interest are bacteria, which may be Gram-positive or Gram-negative, or Gram-indeterminate or Gram-non-responsive, and fungi, e.g. yeast.

The bacteria may aerobic or anaerobic. The bacteria may be, or may include, mycobacteria.

Particularly clinically relevant genera of bacteria include Staphylococcus (including Coagulase-negative Staphylococcus), Clostridium, Escherichia, Salmonella, Pseudomonas, Propionibacterium, Bacillus, Lactobacillus, Legionella, Mycobacterium, Micrococcus, Fusobacterium, Moraxella, Proteus, Escherichia, Klebsiella, Acinetobacter, Burkholderia, Enterococcus, Enterobacter, Citrobacter, Haemophilus, Neisseria, Serratia, Streptococcus (including Alpha-haemolytic and Beta-haemolytic Streptococci), Bacteroides, Yersinia and Stenotrophomonas, and indeed any other enteric or coliform bacteria. Beta-haemolytic Streptococci include Group A, Group B, Group C, Group D, Group E, Group F, Group G and Group H Streptococci.

Non-limiting examples of clinically-relevant Gram-positive bacteria include Staphylococcus aureus (including methicillin-resistant Staphylococcus aureus, MRSA), Staphylococcus haemolyticus, Staphylococcus epidermidis, Staphylococcus saprophyticus, Staphylococcus lugdunensis, Staphylococcus schleiferi, Staphylococcus caprae, Streptococcus salivarius, Streptococcus agalactiae, Streptococcus anginosus, Streptococcus pneumoniae, Streptococcus pyogenes, Streptococcus sanguinis Streptococcus mitis, Streptococcus equinus, Streptococcus bovis, Clostridium perfringens, Clostridium sordellii, Clostridium novyi, Clostridium botulinum, Clostridium tetani, Enterococcus faecalis, and Enterococcus faecium. Non-limiting examples of clinically-relevant Gram-negative bacteria include Escherichia coli, Salmonella bongori, Salmonella enterica, Citrobacter koseri, Citrobacter freundii, Klebsiella pneumoniae, Klebsiella oxytoca, Pseudomonas aeruginosa, Haemophilus influenzae, Neisseria meningitidis, Enterobacter cloacae, Enterobacter aerogenes, Serratia marcescens, Stenotrophomonas maltophilia, Morganella morganii, Bacteroides fragilis, Acinetobacter baumannii and Proteus mirabilis.

Non-limiting examples of clinically-relevant fungi include yeasts, particularly of the genus Candida, and fungi in the genera Aspergillus, Fusarium, Penicilium, Pneumocystis, Cryptococcus, Coccidiodes, Malassezia, Trichosporon, Acremonium, Rhizopus, Mucor and Absidia. Of particular interest are Candida and Aspergillus, including Aspergillus fumigatus, Candida albicans, Candida tropicalis, Candida glabrata, Candida dubliensis, Candida parapsilosis, and Candida krusei.

Non-limiting examples of clinically-relevant protozoa include Entamoeba histo/ytica, Giardia lamblia, Trypanosoma brucei, Besnoitia besnoiti, Besnoitia bennetti, Besnoitia tarandi, Isospora canis, Eimeria tenella, Cryptosporidium parvum, Hammondia heydorni, Toxop/asmosa gondii, Neospora caninum, Hepatozoon canis, Plasmodium falciparum, Plasmodium vivax, Plasmodium ovale, Plasmodium malariae and Plasmodium knowlesi.

The term “mammalian cell” encompasses any cell of mammalian origin. The cell may originate from any mammal, particularly a human (i.e. it may be a human cell). The cell may originate from a domestic animal, e.g. a farm animal such as a horse, donkey, sheep, pig, goat or cow, or an animal commonly kept as a pet such as a cat, dog, mouse, rat, rabbit, guinea pig or chinchilla. The cell may be any type of cell. In particular embodiments the cell is a blood cell, e.g. a red blood cell (erythrocyte) or a white blood cell (leukocyte), such as a neutrophil, monocyte or lymphocyte. A platelet is considered herein a blood cell.

The sample comprising microorganisms and mammalian cells may, as noted above, be any such sample, but is, or is derived from, in particular a clinical sample or veterinary sample. A clinical sample is any sample obtained from a human. It may thus be any sample of body tissue, cells or fluid, or any sample derived from the body, e.g. a swab, wash, aspirate or rinse etc. Suitable clinical samples include, but are not limited to, samples of blood, serum or plasma, blood fractions, joint fluid, urine, semen, saliva, faeces, cerebrospinal fluid, gastric contents, vaginal secretions, mucus, a tissue biopsy sample, tissue homogenates, bone marrow aspirates, bone homogenates, sputum, respiratory samples, wound exudate, swabs and swab rinsates, e.g. a nasopharyngeal swab, other bodily fluids and the like. In a preferred embodiment, the clinical sample is sample is blood or a blood-derived sample, e.g. serum or plasma or a blood fraction. A veterinary sample is an equivalent sample derived from a non-human animal, in this case a non-human mammal. As discussed further below, the sample may also be a culture of a clinical or veterinary sample, e.g. a blood culture.

The nature of the clinical or veterinary sample may be determined according to the presentation of symptoms of the infection or suspected infection, or the general clinical condition of the subject. Although any microbial infection is encompassed, the method of the invention has particular utility in the course of detection or diagnosis of sepsis (or more generally management of sepsis), or where sepsis is suspected. Thus the clinical or veterinary sample may be from a subject having, or suspected of having, or at risk of, sepsis. In such a case the sample will generally be blood or a blood-derived sample. Typically, for sepsis the sample will be, or will comprise, blood, but it is not precluded that other types of sample, such as those listed above.

The clinical sample may be introduced to a culture vessel comprising culture medium. This is a standard step which may be carried out according to standard procedures well known in the art and widely described in the literature. The clinical sample may thus be subjected to culture and thus the sample used in the method may a culture of a clinical sample (or correspondingly a veterinary sample). The following discussion is made in the context of a clinical sample, but it will be understood that this may refer analogously to a veterinary sample.

A clinical sample may be collected in a vessel containing culture medium suitable for culturing microbial cells. It may in some embodiments be desirable to introduce a clinical sample into a culture flask and immediately or after only a short period of culture to remove an aliquot of the clinical sample/culture medium mixture from the flask for testing (e.g. for microbial ID), whilst subjecting the culture flask to continued culture, before further testing (e.g. AST testing). Such a method is described in WO 2015/189390.

A culture vessel can include any vessel or container suitable for the culture of microbial cells, e.g. a plate, well, tube, bottle, flask etc. Conveniently, where the clinical sample is blood or a blood-derived sample the culture vessel is a blood culture flask, or indeed any tube, flask or bottle known for the sampling of blood, particularly for the purpose of culture to detect microorganisms. The sample may, therefore, be a blood culture sample.

Conveniently the culture vessel may be provided with the culture medium already contained therein. However, the culture medium may be separately provided and introduced into the culture vessel, either prior to, simultaneously with, or after the clinical sample has been added.

The culture medium may be any suitable medium and may be selected according to the nature of the clinical sample and/or the suspected microorganism, and/or clinical condition of the subject from whom the sample is derived, etc. Many different microbial culture media suitable for such use are known. Typically the culture medium may contain sufficient nutrients to promote rapid growth of microorganisms, as is known in the art. In many cases appropriate media are complex growth media comprising media such as Muller-Hinton (MH) media, MH—fastidious (MHF), Muller-Hinton supplemented with lysed horse blood, Lysogeny broth (LB), 2× YT Media, tryptic soy broth (TSB), Columbia broth, brain heart infusion (BHI) broth and Brucella broth, as well as general purpose growth media known in the art, and may include the addition of particular growth factors or supplements. The culture may or may not be agitated. Culture media are available in various forms, including liquid, solid, and suspensions etc. and any of these may be used, but conveniently the medium will be a liquid medium. Where the culture vessel is a ready-to-use blood culture flask, as described above, these vessels may contain specified media especially modified to allow a wide range of microorganisms to grow. Typically medium supplied in a blood culture flask by a manufacturer will contain an agent or additive to neutralise the presence of any antibiotics present in a clinical sample taken from a test subject. Flasks containing or not containing such neutralising agents may be used, and neutralising agents may be added to the culture vessel if desired.

In a particular aspect of the present invention, the clinical sample is blood or a blood-derived sample, and is collected in a blood culture flask (BCF). Examples of blood culture flasks include a BacT/ALERT (Biomerieux) blood culture flask, a Bactec blood culture flask (Becton Dickinson) or VersaTrek blood culture flask (Thermo Fisher), or indeed any tube, flask or bottle known for the sampling of blood, particularly for the purpose of culture to detect microorganisms.

Such a blood culture flask etc. may contain a resin, and the method may accordingly comprise a step of removing the resin from the sample, e.g. by filtering. For example such a resin pre-filtration step may be performed before carrying out step (b) of the method.

A sample according to the invention may accordingly comprise a clinical sample in a culture medium. Further the sample may be a clinical sample culture (i.e. a clinical sample which has been cultured for a period of time). It will be seen in this respect that the sample which is subjected to the method of the invention may be a portion of a complex sample which is collected or prepared. Thus the sample of the method of the invention may in one embodiment be an aliquot (e.g. a test aliquot) taken or removed from the sample e.g. from the contents of a culture vessel (flask) containing a clinical or other sample, whether before, during or after a period of culture (i.e. incubation).

In one embodiment, therefore, the sample provided in step (a) may be a culture of a clinical sample which has been designated as positive for microbial growth (e.g. in a clinical sample culture system). Thus it may be a positive blood culture flask. However, it is not necessary according to the methods of the present invention for the clinical sample culture to be designated as positive and such a clinical culture sample may be used at a stage before it has been designated as positive, e.g. when it has been cultured for a period of time less than that necessary for it be indicated as positive. Thus the sample may be a non-positive blood culture flask (e.g. a blood culture flask which contains fewer microbial cells than is required for the flask to be designated as positive, or which has been cultured for a shorter period of time). Indeed, in the case of some clinical samples, a sample of the clinical sample culture may be withdrawn and used in the methods of the invention before any culture has taken place (e.g. when the clinical sample culture is set up).

It is known that certain microorganisms are difficult to culture, and that in a clinical context such microorganisms may not be detected in traditional or conventional methods clinical detection or diagnostic methods based on a culture step. For example certain bacteria are difficult to grow on solid media, which are commonly used in diagnostic methods. Thus, the number of clinically relevant microorganisms may far exceed those which are typically tested for and analysed today. Such “unculturable” microorganisms (e.g. bacteria) for which standard culture methods may not yet be available may be grown in certain liquid media, for example with various supplements or additives, for example sera or other blood components or BHI etc. However, such supplements or additives may interfere in the concentration determination methods and may need to be removed. The methods disclosed herein may have applicability in such situations, and the sample may accordingly be a sample of a culture of such a microorganism. The microorganism may be present in a clinical or veterinary sample which has been subjected to culture (for example in a specialist culture medium containing a supplement or an additive). However, in another embodiment disclosed herein the culture may be of an isolate of a microorganism (e.g. an isolate from another culture) and hence in such a situation the sample may not necessarily comprise mammalian cells. Such a sample may be used in the context of a method for determining the concentration of a microorganism in a suspension as disclosed above (i.e. a method which does not include the steps of providing a sample containing microorganisms and mammalian cells and recovering microorganisms therefrom).

In methods comprising the recovery of microorganisms from a sample comprising microorganisms and mammalian cells, the sample is contacted with a buffer solution, a detergent and one or more proteases. The contacting of the sample with these reagents causes lysis of the mammalian cells present in the sample. The reagents cause the lysis of mammalian cells but do not cause lysis of microbial cells. In particular, the reagents do not cause lysis of bacterial cells. Preferably the reagents also do not cause lysis of fungal cells; preferably the reagents also does not cause lysis of non-mammalian eukaryotic microbial cells, e.g. protists. The reagents generally act by solubilising mammalian cell membranes. The selective lysis of non-microbial cells allows the microbial cells to be separated from other components that may be present in the sample. The term “lysing” means breaking down of a cell. In particular, the cell is broken down to release cell contents. The term “selectively lysing” or “selective lysis” means lysing of a particular subset of the cells present in a sample. In the present case it is desirable to selectively lyse only the non-microbial cells, or more particularly the cells which derive from the subject under test (e.g. mammalian cells) that are present in a clinical or veterinary sample, without substantially lysing the microbial cells present in a clinical or veterinary sample. In addition, it is desirable according to certain methods of the present invention that the microbial cells obtained from the sample are able to grow and reproduce (growth is required in order to determine antimicrobial susceptibility), and thus it is desirable that the ability of the microbial cells to grow and/or reproduce (viability) is not affected by the selective lysis of the non-microbial or test subject-derived cells that are present in a sample.

Preferably all (i.e. 100%) or substantially all of the microbial cells present in the sample remain intact, or more particularly, viable, following selective lysis of the mammalian cells, and it is preferred that at least 99%, 98%, 97%, 96%, 95%, 94%, 93%, 92%, 91% or 90% of microbial cells in the sample remain intact, or viable, following the selective lysis step. However, as the methods of the present invention require the concentration of intact or viable microorganisms in the recovered microorganism sample to be determined, antibiotic susceptibility may still be assessed in the event that at least 80%, 70%, 60%, 50%, 40%, 30%, 20% or 10% of the microbial cells remain viable. Thus, such methods are not limited to any particular level of microbial viability following selective lysis of the mammalian cells.

The buffer solution has a pH of at least pH 6 and at most pH 9, i.e. the buffer solution has a pH in the range of pH 6 to pH 9. In particular embodiments the buffer solution has a pH in the range pH 6.0 to pH 8.5, pH 6 to pH 8, pH 6.5 to pH 8.0 or pH 7 to pH 8. Optimally the buffer solution has a pH of about 7.5.

The buffer solution may comprise chaotropes or chaotropic agents to increase target cell (i.e. mammalian cell) lysis, e.g. urea, guanidinium hydrochloride, lithium perchlorate, lithium acetate, phenol, or thiourea. In certain embodiments, however, the buffer solution does not comprise a chaotrope or chaotropic agent. In particular embodiments, no such agent may be used during the course of the recovery of microorganisms from a sample (and more particularly is not used during a selective lysis step), and/or during the course of the concentration determination method of the present invention.

The buffer solution preferably does not comprise an alcohol. The buffer solution may further comprise reducing agents (e.g. 2-mercaptoethanol or dithriothreitol (DTT)), stabilising agents (e.g. magnesium or pyruvate), humectants and/or chelating agents (e.g. ethylenediaminetetraacetic acid (EDTA)).

Additionally, the buffer solution may comprise any suitable salts, including NaCl, KCl, MgCl₂, KH₂PO₄, K₂HPO₄, Na₂HPO₄ and NaH₂PO₄. Such salts might aid mammalian cell lysis or the subsequent handling of the microbial cells. Salts may, if present, be present at any suitable concentration, e.g. at least 0.01 M, 0.02 M, 0.05 M, 0.1 M, 0.2 M, 0.5 M, 1 M, 2 M or 5 M, depending on the factors such as the volume of buffer and sample used.

In a particular embodiment, the buffer solution is a PBS (phosphate-buffered saline) buffer. PBS comprises disodium hydrogen phosphate (Na₂HPO₄), NaCl, and optionally KCl and/or monopotassium phosphate (KH₂PO₄). PBS may be acquired from a manufacturer, e.g. Sigma-Aldrich or Thermo Fisher Scientific, or may easily be made from its constituent parts. An exemplary recipe for 1×PBS is NaCl 137 mM, KCl 2.7 mM, Na₂HPO₄ 10 mM, KH₂HPO₄ 1.8 mM; the pH may be adjusted up or down with NaOH or HCl, respectively.

The buffer solution added to the sample may be at a higher concentration than its concentration for use, e.g. the buffer solution added may be 5× or 10× concentration, so that on mixing with the sample it is diluted to its concentration for use.

The detergent may be an ionic detergent, non-ionic detergent or zwitterionic detergent. An ionic detergent carries an electrical charge, which may be positive (cationic detergents) or negative (anionic detergents). Zwitterionic detergents possess multiple charged groups; generally zwitterionic detergents have the same number of positive and negative charges and so have a net zero charge. Non-ionic detergents have uncharged, hydrophilic headgroups.

Exemplary ionic detergents which may be used include alkylbenzenesulfonates, N-lauroylsarcosine, deoxycholic acid (or a salt thereof e.g. sodium deoxycholate), cetrimonium bromide (CTAB) and sodium dodecyl sulphate (SDS).

Examplary zwitterionic detergents which may be used include CHAPS, sulfobetaines (e.g. SB 3-10 and SB 3-12), amidosulfobetaines (e.g. ASB-14 and ASB-16) and C7BzO.

Preferably, the detergent is a non-ionic detergent. Exemplary non-ionic detergents which may be used include the Triton detergent series, e.g. Triton X100-R and Triton X-114, NP-40, Genapol C-100, Genapol X-100, Igepal CA 630, Arlasolve 200, the Brij detergent series, e.g. Brij-O10, Brij-97, Brij-98, Brij-58 and Brij-35, octyl β-D-glucopyranoside, polysorbates, e.g. polysorbate 20 and polysorbate 80 and the Pluronic detergent series, e.g. Pluronic L64 and Pluronic P84. In one embodiment polyoxyethylene detergents may be used. The polyoxyethylene detergent can comprise the structure C12-18/E9_10, wherein C12-18 denotes a carbon chain length of 12 to 18 carbon atoms and E9-10 denotes from 9 to 10 oxyethylene hydrophilic head groups. In a particular embodiment the detergent is Brij-O10, which may be obtained from e.g. Sigma-Aldrich (product P6136). Brij-O10 has the chemical formula:

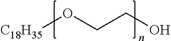

wherein n is about 10, preferably 10.

The detergent is added to a suitable resultant concentration. Such a concentration is known to the skilled person or may be identified for any selected detergent by routine optimisation. In a particular embodiment, the detergent is contacted with the sample at a concentration (i.e. resultant concentration following addition of the detergent to the sample) of from 0.1% to 5% w/v, for instance between 0.1% and 1% w/v. In a particular embodiment, the detergent is contacted with the sample at a concentration of about 0.45% w/v.

The protease may be any suitable protease. It may be an endopeptidase or an exopeptidase, and it may use any proteolytic mechanism, e.g. it may be a serine protease, cysteine protease, aspartyl protease, metalloprotease, etc. Exemplary protease enzymes which may be used in the method of the invention include Type XXIII proteinase, proteinase K, pepsin, trypsin, chymotrypsin, papain, elastase and cathepsins. Preferably the protease is an endopeptidase. In a particular embodiment the protease is proteinase K. The skilled person is able to determine an appropriate concentration of protease to use in the method of the invention, depending on the sample, the protease used, etc. For instance, proteinase K may be used at a final concentration in the range of 20 to 200 μg/ml, e.g. 50 to 150 μg/ml or 50 to 100 μg/ml. Preferably, proteinase K is used at a final concentration of about 50 to 80 μg/ml.

The sample may also be contacted with additional enzymes to aid mammalian cell lysis in step (b), e.g. nuclease enzymes such as DNase or RNase, lipase, glycoside hydrolases such as neuraminidase, amylase, etc.

In step (b), the sample may be contacted separately with the buffer solution, detergent and at least one protease. Alternatively the three components (buffer, detergent, protease) may be prepared (e.g. pre-prepared as a combined composition, or prepared in use) in one or more combinations before contact with the sample. The term “contacting” is used broadly herein to include any means of contacting the sample with the reagent, in any order. Thus, the sample may be added to the component (e.g. a component already present in a reaction vessel) or the component may be added to the sample (e.g. a sample already present in a reaction vessel). The three, or any two of the three components may be pre-prepared as a combined composition to be contacted with the sample, or the components may be added (e.g. to a reaction vessel) sequentially, prior to contact with the sample. In a preferred embodiment, the detergent is provided in a lysis buffer, comprising the detergent dissolved in the above-described buffer solution. The at least one protease may then be added to the lysis buffer, and the resulting composition added to the sample (or vice versa), such that the sample is contacted simultaneously with the buffer solution, detergent and protease. In a particular embodiment the lysis buffer comprises PBS pH 7.5, 0.45% w/v Brij-010. In a particular embodiment, the sample is contacted with a composition comprising: (i) the lysis buffer comprising PBS pH 7.5 and 0.45% w/v Brij-O10 and (ii) proteinase K.

The contacting of step (b) (i.e. the contacting (or incubation) of the sample with the buffer solution, detergent and one or more proteases) is performed for a suitable period of time. For instance, the contacting may take place for up to 1 hr, e.g. up to 30 mins, up to 20 mins or up to 10 mins. The contacting is performed at a suitable temperature, e.g. at least 4° C., for instance 20-40° C., e.g. 25-37° C. The aliquot may be heated for 5-20 mins, preferably 5 to 10 mins.

The mixture obtained in step (b) is filtered. The filtration process allows separation of the intact microbial cells and the products of the mammalian cell lysis, and optionally any other debris or material present in the sample. The intact microbial cells are caught within the filter while the products of the mammalian cell lysis pass through for disposal, thus removing the lysed mammalian cells from the suspension. Filtration is performed using a filter comprising a suitable pore size to capture any microbial cells. The filter may have a pore size of 0.5 μm or less; preferably the filter has a pore size of 0.25 μm or less. The filter may be made of any suitable material, e.g. many appropriate filters are made of PTFE (polytetrafluoroethylene). Suitable filters may be commercially purchased, e.g. from Merck. In some embodiments, the filter used has a large surface area relative to the volume of sample filtered through it, to prevent the filter becoming clogged with the microorganisms. For example the filter may have a size range of 30-100, 30-80 mm or 30-75 mm (e.g. 50 mm). However, filters of any size may be used, e.g. in the range of 4-100, 4-80 or 4-75 mm. This may depend on the nature of the sample and the amount of microorganisms in the sample. For example, a positive blood culture may contain many more microorganism than a clinical urine sample and it may be beneficial to use a larger filter size. An appropriate filter size can be determined by routine trial and error.

Following filtration, the isolated microbial cells (i.e. those caught on or within the filter) may be washed to remove residual lysis buffer, mammalian cell debris, etc. Washing, if performed, takes place between steps (c) and (d). Washing may be performed by flushing wash buffer through the filter. The filter may be washed with any appropriate wash buffer, as known to the skilled person. Suitable wash buffers include e.g. a buffer solution as described above, such as PBS. In a particular embodiment the wash buffer may be a buffer solution as described above, and in certain embodiments may be the same as the buffer solution used in step (b). However, in other embodiments, the wash buffer may comprise a protease (and optionally not a detergent) or a detergent (and optionally not a protease). In certain embodiments the wash buffer may comprise a chaotrope, whereas in other embodiments it may not comprise a chaotrope, e.g. as described above. In yet further embodiments, the wash buffer may be a culture medium, as described above. In a particular embodiment, the wash buffer is cation-adjusted Mueller Hinton Broth (CAMHB), which may be purchased from e.g. Sigma-Aldrich. CAMHB is alternatively known as a Mueller Hinton Broth 2. The filter (including the isolated microbial cells) may be washed one or more times, as required to remove mammalian cell debris from the filter, e.g. the filter may be washed 2, 3, 4 or 5 or more times.

Following filtration and optional washing, the microbial cells are recovered from the filter. Recovery of the microbial cells comprises resuspending the cells in a liquid, thus providing a suspension of the recovered microorganisms. The cells may be resuspended from the surface of the filter by repeated pipetting using the liquid. In one preferred embodiment of the invention liquid is back-flushed through the filter (i.e. in the opposite direction to which the filtrate was filtered) in order to resuspend the microbial cells. In another embodiment, the microorganisms are recovered in the last fraction of the wash solution that is drawn back through the filter. Alternatively microbial cells may be retrieved by using the entire filter, e.g. either by adding liquid to the filter or contacting the filter with liquid in a vessel.

The liquid in which the microbial cells are resuspended may be any suitable liquid, e.g. buffer or culture medium. In a preferred embodiment the microbial cells are resuspended in culture medium (that is to say, a liquid growth medium suitable for culturing microorganisms). When culture medium is used to resuspend the microbial cells, the culture medium is generally speaking a culture medium which is approved or recognised for use in AST assays. In one embodiment it is a Muller-Hinton (MH) medium or a Muller-Hinton Fastidious (MHF) medium, or cation-adjusted Mueller Hinton medium (CAMHB). For non-standard AST any other medium commonly known may be used with the invention. MIC values obtained by performing an AST assay using a ‘non-standard’ culture medium may be adjusted (correlated) to give standard AST results. In other embodiments the resuspension liquid may be PBS, or other buffer. In further embodiments, the resuspension liquid is not water (e.g. tap water, ground water or sterilised water). Furthermore, in particular embodiments, the liquid in which the microbial cells are resuspended may not comprise a proteolytic enzyme, such as papain, trypsin, a neutrase, subtilisin or a subtilisin-like enzyme, or Rhozyme.

Once the microbial cells have been recovered and the recovered microorganism sample has been obtained, the concentration of microbial cells present in the recovered microorganism sample is determined according to the methods of the present invention. In one particular embodiment, as noted above, this may be in particular with a view to performing an AST assay, i.e. the concentration of microorganisms may be determined before an AST assay is performed.

Advantageously, performing an AST assay using a recovered microorganism sample may allow a more rapid AST assay to be performed. In particular, by recovering microbial cells directly from a clinical sample or clinical sample culture, thereby to obtain a recovered microorganism sample, a homogeneous sample lacking any contaminants is provided.

Certain samples, e.g. food or environmental samples in particular, may comprise particulate matter which it may be desirable to remove prior to determining the concentration of intact microorganisms in a sample. Additionally, certain commercially-available culture vessels (e.g. blood culture flasks) are provided with resin beads, which resin neutralises the effect of any antimicrobial agents which are present in the clinical sample (i.e. which had been administered to the subject under test) in order to facilitate the growth of the microbial cells in culture. In a preferred embodiment, therefore, the sample may be filtered to remove any large particles that may be present in the sample. Preferably, this step of filtration will utilise a filter having a pore size which does not substantially remove any cellular matter from the test aliquot, but which can remove the particles, e.g. at least 100, 200 or 300 μm, but could be up to 1000 μm. Such a filtration step may take place at any point in the method of the present invention. In particular embodiments, such a step may take place prior to imaging the suspension-stain mixture in step (e)(iv) in order to avoid any such particles being imaged. Thus, such a step may take place prior to step (e)(iii) or step (e)(i), and more particularly may take place prior to step (e). More particularly, such a step may take place prior to step (c) or step (b), and yet more particularly may take place prior to step (a). In certain embodiments, the sample provided in step (a) may have been subjected to such a filtration step in order to remove particulate matter. In order to determine the concentration of intact microbial cells in the suspension, the suspension is first aliquoted, that is to say it is divided into one or more smaller portions/samples. An aliquot (i.e. portion) of the suspension is first treated (in step (e)(i)) to enhance the staining process. The treatment (or “pre-treatment”) step may comprise contacting the aliquot with an alcohol, for example with ethanol. Other suitable alcohols include methanol, propanol, isopropanol, butanol (of any isomeric form), etc. The skilled person is able to select an appropriate alcohol. In a preferred embodiment the aliquot is contacted with ethanol. In certain embodiments, the aliquot is contacted with alcohol to provide a mixture comprising 25-45% v/v alcohol, e.g. 25-35% v/v alcohol, 30-40% v/v alcohol or 30-35% v/v alcohol (e.g. ethanol). In a particular embodiment, the aliquot is contacted with alcohol to provide a mixture comprising 30% v/v alcohol (e.g. ethanol). In another particular embodiment, the aliquot is contacted with alcohol to provide a mixture comprising 35% v/v alcohol (e.g. ethanol).

In an alternative embodiment, the treatment step comprises heating the aliquot of the suspension. The aliquot may be heated to a temperature in the range of 50-90° C., for instance 60-80° C. or 65-75° C. In a particular embodiment the aliquot is heated to a temperature of about 70° C. The aliquot may be heated for an amount of time appropriate for the temperature used, i.e. the higher the temperature selected, the shorter the heating time required (and vice versa). In an embodiment the aliquot is heated for from 30 seconds up to 20 mins, or up to 10 mins. The aliquot may thus be heated for 0.5-20 or 0.5-15, or 0.5-10 minutes (time measured as the time at the relevant temperature, i.e. not ramping times). The skilled person is able to select an appropriate heating time for a given heating temperature.

Heating may be performed in e.g. an incubator, a heat block, an oven, a thermal cycler or any other suitable means.

In certain embodiments treatment with an alcohol may be combined with heat treatment step, simultaneously or separately (e.g. sequentially).

In another alternative embodiment, the treatment step comprises contacting the aliquot of the suspension with a detergent. Suitable detergents are described above in relation to the lysis buffer of step (b). When the suspension of microbial cells is contacted with a detergent in step (e)(i), a detergent as described in step (b) may be used, but at a much higher concentration than it was used in step (b). Thus while the detergent in the buffer solution of step (b) may be present at a concentration of e.g. 0.1% to 5% w/v, for instance between 0.1% and 1% w/v, as described above, the detergent used in step (e)(i) is used at a much higher concentration than this, preferably 5-20 times higher, e.g. 10 times higher. The detergent may be used in step (e)(i) at a concentration of 0.5% to 50% w/v, preferably 1% to 10% w/v, e.g. about 5% w/v.

In embodiments where the aliquot of the suspension is treated with an alcohol or a detergent, the treatment may take place at or around room temperature, e.g. the treatment may take place at a temperature in the range 20-37° C., e.g. 20-30° C., 25-30° C. or 30-35° C. Alternatively, as noted above, this may be combined with a heating step. The contacting may be performed by way of an incubation at the chosen temperature with the chosen concentration of alcohol or detergent. The incubation may last from 30 seconds up to 1 hr, e.g. up to 30 mins, up to 20 mins, up to 10 mins or up to 5 mins. The precise time will depend on the sample, the microorganisms which are present in the sample, and/or whether or not a heat treatment step is include. In a preferred embodiment, the incubation lasts for from 5 to 10 mins, preferably about 5 mins.

In certain embodiments, the treatment step does not comprise contacting the sample with an aldehyde or a ketone. In particular, the treatment step may not comprise contacting the sample with formaldehyde, ethanol, propanal, propanone, butanal or butanone. In yet further embodiments, the treatment step does not comprise contacting the sample with a carboxylic acid, such as methanoic acid, ethanoic acid, oxalic acid, propanoic acid, malonic acid, butanoic acid or succinic acid. In further particular embodiments, the treatment step does not comprise contacting the sample with an aldehyde, ketone or carboxylic acid (e.g. as listed above) in combination with a heat treatment step, and/or in combination with contacting the sample with an alcohol and/or a detergent. In yet further embodiments, the treatment step does not comprise contacting the sample with an antibiotic, in particular an antibiotic which may allow bacterial growth but which may inhibit cell division, such as chloramphenicol and penicillin such as ampicillin, benzyme penicillin, cloxacillin, dicloxacillin, or combinations thereof.

A sample analysed by the method of the invention may contain a wide range of possible different concentrations of microorganisms, and it may not be possible for a single calibration curve to be prepared in order to allow such a range of concentrations to be accurately determined. It may, therefore, be beneficial to dilute the aliquot of the sample containing microorganisms during the course of performing the method of the present invention, such that the image analysis value for the number of objects determined in step (e)(iv) falls within the range of a pre-determined calibration curve.

Further, depending on the nature of the suspension and/or treatment, it may be desirable to dilute the sample (that is the aliquot of the suspension taken to allow concentration determination in step (e)) to allow the concentration determination to be performed, e.g. to dilute (or minimise or reduce the amount of) contaminants or components which may interfere in the concentration determination method. For example, certain media (e.g. Muller Hinton media) contain components which may interfere in fluorescence determinations, and if the sample is a culture sample containing such media, or if recovered microorganisms are resuspended in such a medium, then a dilution step may be desirable. Similarly, if the treatment is performed using an alcohol or a detergent, a dilution step may be desirable. Alternatively, if microorganisms are resuspended from the filter in a buffer, e.g. PBS, a dilution step, or more particularly an initial dilution step may not be necessary. This may be relevant in the context of a method where microorganisms are present in the suspension at low concentration (in low amounts), where in such situations it may be desirable to resuspend the recovered microorganisms in a buffer such as PBS.

When a dilution is to be made, i.e. where an aliquot of the sample is diluted in step (e)(ii) to provide a diluted aliquot at a dilution value, such a dilution may be performed before, during or after step (i). An aliquot of the sample may, therefore, be diluted prior to being contacted with the stain, either before, during or after treatment in step (e)(i). In such a situation the dilution medium may be a buffer, or saline or water or other aqueous solution etc., as is discussed in further detail below.

In an embodiment, a dilution before step (e)(i) is not performed (i.e. there is no dilution before contacting with the alcohol (or detergent) or heat). In other words, the dilution may take place during or after step (e)(i).

In another embodiment, where the “pre-treatment” of step (e)(i) involves contacting with alcohol or detergent, the contacting may itself provide a dilution step. This can be seen as a step of dilution during step (e)(i).

In other embodiments, the methods herein may comprise a dilution step which is performed after the contacting/heating of step (e)(i), for example after contacting with alcohol.

In a particular embodiment, the methods may comprise performing the dilution of step (e)(ii) during and after step (e)(i). For example, a dilution of the aliquot may take place during step (e)(i), during the contacting with alcohol, and a further dilution may take place after the contacting with alcohol.

Two or more aliquots may be prepared, such that each aliquot is diluted to different extents. In other words, each aliquot may be diluted at a different dilution factor or dilution value. In such an embodiment, a first aliquot (i.e. at a first dilution value) may be an aliquot of the sample, and a second aliquot (or subsequent) aliquot may be a diluted aliquot at a second (or subsequent) dilution value. Alternatively, two separate dilutions may be performed. One or more of the diluted aliquots may be diluted by serial dilution. Thus, a dilution series may be prepared, by a set of sequential, separate or simultaneous steps, as desired.

When the aliquot is treated in step (e)(i) using heat, if dilution of the suspension is desired this may be performed before, during or after heating (i.e. the dilution of step (e)(ii) may be performed before, during or after the treatment step of (e)(i)). However, if an alcohol or detergent is used to treat the aliquot, it is in one embodiment preferred that dilution of the aliquot is performed after the treatment step of (e)(i), to dilute the alcohol or the detergent and thus enhance the staining/imaging process. In particular, ethanol may interfere with the staining process of the claimed method, and so it is preferred that if ethanol is used for treatment of the aliquot of suspension, it is diluted prior to imaging to lower the ethanol concentration.

In certain embodiments of the invention, where two or more aliquots are prepared, each said aliquot may be prepared simultaneously (or substantially simultaneously, including by sequential or serial steps) before step (e)(iii) of contacting with the stain. In such an event, steps (e)(iv) and (e)(v) may be performed on each aliquot simultaneously or sequentially. In other words, each aliquot may be imaged simultaneously (i.e. in parallel), or sequentially, and the respective image analysis values obtained from each aliquot may be compared to a pre-determined calibration curve. Steps (e)(iv) and (e)(v) may alternatively be performed on a first aliquot, and if the image analysis value obtained from said aliquot falls within the range of a pre-determined standard calibration curve, steps (e)(iv) and (e)(v) may be dispensed with for second or further aliquots. An aliquot may be the or an aliquot of the treated suspension of step (e)(i) or of a diluted aliquot of step (e)(ii).

In an alternative embodiment, however, a diluted aliquot (or second or further diluted aliquot) may only prepared once the steps of the method have been performed on a first aliquot (which may be the or an aliquot of the treated suspension of (e)(i) or a diluted aliquot of (e)(ii)). Such an embodiment may be desirable if, for example, the image analysis value does not fall within the range of a pre-determined calibration curve. In such an embodiment, it may be necessary for the method of the invention to be repeated on a second (or further) aliquot at a different dilution value. In such an event, it will be seen that each of the two (or further) aliquots are prepared sequentially, i.e. after steps (e)(iv) and/or (e)(v) have been performed e.g. on a first aliquot.

Steps (e)(iv) and/or (e)(v) may therefore be performed on one aliquot (which may be a pre-treated, but diluted or non-diluted aliquot), even if more than one aliquot is prepared, or on two more aliquots (which may be diluted aliquots, or may include an undiluted aliquot).

The steps (e)(iii) and (e)(iv) may therefore be performed on each aliquot of two or more aliquots, thereby to determine an image analysis value for the number of objects corresponding to viable microorganisms in each aliquot. Where two or more image analysis values have been obtained for each of two or more aliquots, step (e)(v) may comprise identifying an aliquot which comprises an image analysis value within the range of a pre-determined calibration curve, and comparing the image analysis value for said aliquot to a pre-determined calibration curve, thereby to determine the concentration of microorganisms in said sample. In such an event, steps (e)(iii) and (e)(iv) may be performed on each aliquot sequentially or simultaneously. As noted above, the aliquots may be diluted aliquots, or they may comprise an undiluted aliquot.

Dilution may comprise contacting an aliquot of the sample with a volume of a suitable sterile buffer or aqueous solution (e.g. saline or a salt solution) or indeed any suitable diluent. The aliquot may be diluted using the same liquid used to form the suspension of microorganisms in step (d), e.g. a culture medium. Preferably a buffer is used to dilute the aliquot of the suspension. The buffer may be any buffer known in the art, e.g. PBS, HBS (HEPES-buffered saline), a Tris buffer such as Tris-HCl or TBS (Tris-buffered saline) or MOPS buffer. In a preferred embodiment, the aliquot of suspension is diluted with PBS.

If the aliquot was treated in step (e)(i) with heat or an alcohol, the diluent may comprise a detergent. The detergent may be as described above with respect to the lysis buffer of step (b), both in terms of the identity and concentration of the detergent. Use of a low concentration of detergent in the diluent aids in calculating the concentration of the microorganism by separating bacterial clusters, thus aiding image analysis.

The treated and optionally-diluted aliquot of suspension is then contacted with a stain, thus providing a suspension-stain mixture. The stain used in the methods of the present invention is a fluorescent stain capable of binding to DNA. The stain may be cell-permeable or cell-impermeable. By “cell-permeable” is meant an agent able to cross the intact membrane of a viable cell; be “cell-impermeable” is meant an agent unable to cross the intact membrane of a viable cell. Without being bound by theory, it is believed that treatment of the cells in step (e)(i) disrupts their cell membranes (and where relevant, cell walls), without lysing the cells. Accordingly, following treatment, a cell-impermeable stain is able to enter and stain the cells, as, of course, is a cell-permeable stain. The stain, being fluorescent, has an emission wavelength which can be detected using a fluorescence detector, thus enabling the identification of stained cells.

Certain stains capable of binding to DNA are also known to have enhanced fluorescence when bound to DNA compared to when present freely in solution. It is preferable that the fluorescent stain selected displays this property. In other words, in a preferred embodiment, the fluorescence intensity of the stain is enhanced when the stain is bound to DNA. Selection of a stain having this property may help reduce the level of background signal generated during detection at the emission wavelength. In particular, a stain may be selected which has low fluorescence when unbound to DNA (i.e. when free in solution). For example, when free in solution the stain may exhibit less than 50%, or more preferably less than 40, 30, 20 or 10% of the fluorescence, or more preferably less than 10%, e.g. less than 9%, 8%, 7%, 6%, 5%, 4%, 3%, 2%, or 1% of the fluorescence which it exhibits when bound to DNA, or less than 0.9%, 0.8%, 0.7%, 0.6%, 0.5%, 0.4%, 0.3%, 0.2% or 0.1% of the fluorescence.

The stain may have excitation and emission wavelengths in the wavelength 350-700 nm. A range of suitable fluorescent stains having emission wavelengths within this range are commonly known in the art, and exemplary fluorescent stains are described below. The fluorescent stain may be a green-fluorescent stain, i.e. having a peak fluorescence emission intensity at or around light having a wavelength of 510 nm. In a preferred embodiment, the stain is a cell-permeable stain.

Particularly preferred stains, having all of the desirable properties described above include SYTO green fluorescent nucleic acid stains (Molecular Probes). SYTO stains are examples of unsymmetrical cyanine dyes, and unsymmetrical cyanine dyes may therefore preferably be used as stains in the methods of the present invention. Structures of SYTO dyes which are available are provided in US U.S. Pat. Nos. 5,658,751, 6,291,203, 5,863,753, 5,534,416 and 5,658,751. A number of different SYTO stains are available, including SYTO 9, SYTO 11, SYTO 12, SYTO 13, SYTO 14, SYTO 16, SYTO 21 and SYTO 24, which may be of use in the methods of the present invention. Particularly preferred are SYTO 9 and/or SYTO 13, or SYTO BC, which is a mixture of dyes. The SYTO BC stain mixture has an excitation wavelength at 473-491 nm and an emission wavelength at 502-561 nm.

Alternatively, the fluorescent stain may be a cell-impermeable stain, which may be red-fluorescent i.e. having a peak fluorescence emission intensity at or around light having a wavelength of 650 nm. A preferred red-fluorescent stain suitable for use in the methods of present invention is propidium iodide (PI).

The stain may, however, be any fluorescent stain capable of staining nucleic acid. These may include SYBR Green, SYBR Gold, SYBR Green II, PicoGreen, RiboGreen, DAPI, Hoechst 3342, Vybrant dyes etc., or indeed any dye commercially available from ThermoFisher. See for example the dyes mentioned in Section 8.1 (Nucleic Acid Stains) of the Molecular Probes Handbook in the technical reference library available on the Thermo Fisher website (https://www.thermofisher.com/se/en/home/references/molecular-probles-the-handbook/nucleic-acid-dectection-and-genomics-technology/necleic-acid-stains.html), which is incorporated herein by reference.

The aliquot may be contacted with the stain at a temperature which is not harmful to the cells in the suspension-stain mixture, and which allows staining to take place. A suitable temperature may be selected, for example, based on the nature of the sample, the identity of a microorganism therein or the properties of the stain used. However, typically temperatures of 37° C. or less are used, in order to avoid damaging microorganisms in a sample. Thus, temperatures of 35° C., 30° C. or 25° or less may be used. It is also preferred that temperatures of 4° C. or greater are used, for example 5° C., 10° C. or 15° C. or greater. In a preferred embodiment, the sample is contacted with the stain at 20-30° C., more particularly at 20° C.-25° C. In certain embodiments, the sample may thus be contacted with the stain at room temperature.

An object is identified as corresponding to an intact microorganism by detecting a fluorescent signal at the emission wavelength of the stain. Thus, objects corresponding to intact microbial cells have different fluorescence properties to other objects in the sample, and may be distinguished from other objects in the sample (e.g. objects corresponding to non-intact microorganisms, cell debris or other particles present in a sample), thereby to allow the number of objects corresponding to intact microbial cells to be determined. Preferably, only the stained microorganisms corresponding to intact microorganisms in the sample are fluorescent and no other objects are detected during fluorescence imaging.

Imaging of the suspension-stain mixture is performed by visual detection means. A magnified image of the suspension-stain mixture is obtained and analysed to detect objects which correspond to intact microorganisms.

Whilst an object which corresponds to an intact microorganism may be a microbial cell, which may or not be intact after pre-treatment, it may also be a cluster of two or more cells, e.g. a clone growing as a cluster and/or an aggregate of non-clonal cells. Thus, an object may be a microbial cell or cell cluster. Different microorganisms may grow in different ways, e.g. clustering or non-clustering, or with different patterns or morphologies, and for a given microorganism this may also vary depending on the growth conditions, for example the presence or amount of an anti-microbial agent. By analysing the images and counting objects and then correlating the number of objects to a microbial concentration, such different growth patterns and/or morphologies etc., may be taken into account. Thus, the images may be analysed by counting the number of objects and adjusting the number based, for example, on the size and/or intensity of the objects (e.g. to account for clusters or aggregates of cells), to provide an image analysis value for the number of objects, which may then be correlated to the concentration of intact microorganisms using a calibration curve. As noted above, a low concentration of detergent may be added to the sample aliquot to reduce clustering.

Imaging of the suspension-stain mixture may take place at temperatures which are not harmful to microorganisms. Typically this will take place at room temperature, or 20-25° C., although other temperatures, e.g. from at least 4° C. up to 37° C. (i.e. 37° C. or less) may also be used.

Imaging is performed at the emission wavelength of the stain, i.e. to detect objects which are stained by the fluorescent stain. As described above, this provides sufficient information to allow objects corresponding to intact microorganisms to be distinguished from other objects which might be present in the sample.

Imaging may, in addition to fluorescence, comprise the use of microscopy, including brightfield, oblique field, darkfield, dispersion staining, phase contrast, differential interference contrast, confocal microscopy, single-plane illumination, light sheet and/or wide field multiphoton microscopy.

Microorganisms may be allowed to contact, bind, associate with or adsorb onto a detection surface for imaging. However, in a preferred embodiment, imaging is performed on a suspension of microorganisms, i.e. microorganisms which are in a suitable medium or buffer, rather than microorganisms which are attached to, or immobilised on or at a surface. In other words, a volume of the suspension-stain mixture may be imaged. Where imaging is performed on a suspension of microorganisms, an image may be obtained at one or more focal planes through the suspension. It may be preferred for an image to be obtained at two or more (different) focal planes through the suspension (e.g. at different depths or cross-sections through the suspension-stain mixture). In other words, separate sub-volumes of the volume to be imaged may be imaged (i.e. images may be obtained of separate sub-volumes of the suspension-stain mixture volume). Alternatively, images may be obtained at different locations, e.g. different locations in a sample chamber, for example at different X-Y positions in a sample chamber with low height. In such an arrangement most of the microorganisms will be in a single focal plane at each position. Thus, multiple (i.e. two or more) non-overlapping images may be obtained. Such multiple images may include at least 5, 10, 20, 30, 40, 50, 60, 70, 80, 90 or 100 or more images. The images are analysed to detect and/or identify objects corresponding to microorganisms, which as explained above, can be taken to represent or be indicative of intact microorganisms present in the suspension. An image analysis value for the number of objects is thereby obtained. Objects detected in all images obtained of the suspension may provide the total number of objects in the suspension.

To perform the imaging step, the suspension-stain mixture from step (e)(iii), or a portion or aliquot thereof is provided in (e.g. transferred to) a vessel or container in which imaging can take place, for example the well of a plate, or a compartment of a carrier suitable for imaging. Such a well or compartment will have an optical viewing area or space, i.e. a viewing (or viewable) area or space which is accessible to a microscope (or more particularly the objective thereof) and is of optical quality to allow microscopic viewing and imaging. The geometry of the well/compartment may give a viewable area of a defined or desired size (e.g. at least 2 mm by 2 mm), with a suitable or desired liquid height to allow a volume to be imaged (e.g. at least 2 mm liquid height). The objective may be focused on a plane inside the well or compartment, for example parallel to the bottom, removed at a distance from the bottom (e.g. about 0.1-0.5, e.g. 0.2 mm from the bottom), and the microscope may be configured to move the focal plane continuously through the liquid (e.g. upwards through the suspension) during the time of imaging, for example for a total of 1-3 mm (e.g. 1.5 mm) during the image acquisition time (e.g. 10-60, or 20-30 seconds).

In a particularly preferred embodiment, imaging may comprise obtaining a series of 2-D images along an optical axis, wherein each image is obtained at a different position along the optical axis through a volume of the suspension. In certain embodiments, each image may be aligned perpendicularly to the optical axis (here termed xy-aligned). A specific area of the aliquot-sample mixture is covered in a single xy-aligned image the size of which is dependent on the optical properties of the imaging apparatus. For each position in xy-space, one or more 2D images can be collected at different intervals along the optical or z axis. Thus, a series, or stack, of 2D images can be generated, which can, in one embodiment, be used to provide 3D information of a sample volume. Alternatively, multiple individual images providing 2D information can be used. An alternative method of extracting 3D information from a sample is that employed by Unisensor (see e.g. U.S. Pat. No. 8,780,181), where the optical axis is tilted with respect to the xy-plane, and the sample or detector is moved along either the x or y plane. Here, a series of images with an extension into z space, in addition to xy space, is acquired. Through a subsequent transformation of the image data, stacks of 2D images aligned perpendicularly to the xy plane can be achieved also with this method. In this way, each of the series of images is an image of a separate area (separate cross-section), or may alternatively be considered to be a separate volume (a cross section has a defined volume in a z direction, thus a volume comprising the xy space with a depth z may be provided for each image).

Once extracted, the 3D information inherent in the 2D image stacks can be utilized to identify objects corresponding to intact microorganisms in the sample. In one embodiment, 2-D images may be generated from 3-D information by e.g. projections of z-stacks into one 2-D image (a projected 2-D image). Analysis may then be performed using the resulting 2-D image. Alternatively, analysis may be performed on each image obtained through the volume of the suspension, and the results of the analysis may be integrated across all of the 2-D images obtained of the sample. As yet a further alternative, analysis may be performed separately on each of the respective 2-D images obtained (i.e. objects may be determined separately in each 2-D image), and the information gathered therefrom may be combined. Objects may be determined as points or areas of fluorescence intensity indicating an intact microorganism in the field of view under investigation, e.g. in the image or projected 2D image. Analysis may be performed for fluorescent images, and many alternative algorithms for this exist, e.g. in Cellprofiler, and also in most commercial image analysis systems.

In another embodiment, intensity variation in the z space stretching over each position in xy space is registered, indicating microbial mass in a specific position. Integrated over the entire xy space, this gives a measure of total microbial volume. Algorithms for this procedure also exist in commonly-available image analysis software, e.g. in the freeware Cellprofiler.

Alternatively, the microscope can be configured to take images at (e.g. to move the objective to) different locations in the suspension-stain mixture (or field of view), for example in the X-direction (as opposed to the Z-direction).

Once objects corresponding to intact microorganisms (i.e. objects detected at the emission wavelength of the fluorescent stain) have been detected by imaging, the information thus obtained may be used to generate an image analysis value for the aliquot. Images may be analysed for fluorescence intensity and/or size of an (e.g. each) object, and optionally the morphology of an (e.g. each) object. Factors such as the circularity of an object, evenness of fluorescence intensity in an object or maximum fluorescence intensity (e.g. maximum intensity of pixels therein), modal fluorescence intensity, median or mean fluorescence intensity in an object, and/or area of each object detected by imaging may be determined. In certain embodiments, only those objects having one or more of these parameters within a given range may be included in the analysis (e.g. counted or enumerated), thereby to generate an image analysis value. The image analysis value may be a combined value for the objects identified, in the sense of being representative of, or corresponding to, the number of objects, i.e. a count. Object area may be determined on the basis of the number of contiguous pixels contained in each object, and only those objects containing at least or over a certain number of pixels may be included in the analysis. In certain embodiments, objects may be identified and detected on the basis of a derived value for the object area x intensity, and only those objects having properties falling within a particular range of parameters may be counted or enumerated, thereby to generate an image analysis value. In other words, the image analysis value represents the number of objects corresponding to intact microorganisms having characteristics falling within a particular range of parameters, or in other words a corrected (or adjusted) number of objects corresponding to intact microorganisms.

Factors determined for each object (e.g. any of the factors described above) or derived values such as object area x intensity for all of the objects may also be combined to provide information on the population of imaged objects, i.e. on the totality of objects. In this way, for example, maximum, modal or median fluorescence intensity of the imaged objects (or more particularly of a set, or group, of imaged objects) may be determined. Alternatively, the distribution of the fluorescence intensity of the imaged objects, or a derived value such as object area x intensity for the imaged objects may be determined. Thus, each object may have a value assigned to it (e.g. area, maximum fluorescence intensity, total, median or mean intensity), and the median or mean, or variance or standard deviation of one or more of said factors may be established for the population of imaged objects. As described in greater detail below, such information may indicate properties of microorganisms in the suspension, and may be used in the selection of a suitable calibration curve for use in determining the concentration of intact microorganisms therein. Furthermore, such information may provide information on the efficiency of staining of microorganisms in the suspension, and may be used to determine the proportion of microorganisms having a fluorescence intensity below a detection limit.

A background subtraction or normalisation step may optionally be performed for the images as an initial step, i.e. prior to any subsequent image analysis steps described herein. This may be performed using any convenient known standard methods, e.g. rolling ball subtraction.

The image analysis value may be determined after thresholding has been performed. In other words a threshold may be set for determining whether or not an object has been detected. Thresholding may be performed to set a lower limit in the intensity of the signal obtained for an image of the suspension, below which objects are not considered. Within the context of the method of the present invention, thresholding allows objects with a low fluorescence intensity at the emission wavelength (i.e. objects which are not intensely stained with the stain) to be discarded from any future analysis. Thresholds may be set at one or more levels and objects may be counted at different thresholds.

In certain embodiments global thresholding may be performed, i.e. a single threshold value may be set for the whole of an image (or the set of images). In alternative embodiments, however, local thresholding may be performed (e.g. if illumination and/or background signal is not uniform across an image. Local thresholding estimates a threshold value for a given pixel according to the greyscale information of neighbouring pixels.

Further, other image analysis operations may be performed, according to techniques known in the art, prior to determining the image analysis value, for example to convert the image to grayscale (wherein fluorescence intensity may be read as a grayscale level), and/or to subtract background (e.g. using the rolling ball method) etc.

A suspension may be characterised based on information obtained from imaging, for example, whether the microorganisms are clustering or non-clustering microorganisms. Advantageously, selection of a suitable calibration curve for this process may be based only on the appearance of the objects in the suspension, for example whether a particular proportion of the objects detected in the suspension have a particular area and/or maximum intensity, and may not require the identity of the microorganism in said suspension to be known before the concentration of intact microorganisms can be determined by the method of the present invention. A calibration curve may therefore be selected which is predetermined for clustering or non-clustering microorganisms.

The relationship between the concentration of intact microorganisms in a suspension and the image analysis value may depend on a number of parameters regarding the microorganism in said suspension, e.g. the size and morphology of a microorganism, and/or the tendency of a microorganism to form clusters or biofilms. The number of objects in a suspension is therefore not used directly to determine the concentration of intact microorganisms in the suspension, as each object may correspond to two or more microorganisms. Furthermore, a microorganism or a cluster of microorganisms may appear in two separate images if taken at different focal planes in embodiments of the invention where imaging is performed at two or more focal planes, and thus may be detected as two separate objects. Thus, the identity of a microorganism in a suspension may affect the relationship between the concentration of microorganisms in a suspension and the number of objects which are imaged in step (e)(iv) of the methods of the present invention.

Factors such as these, and those previously identified in the art as affecting the accuracy of methods of determining the concentration of viable microorganisms in a suspension (e.g. through imperfect staining of intact microorganisms), may be overcome in the methods of the present invention through the use of calibration curves.

A calibration curve may be prepared by performing steps (e)(iii) and (e)(iv) of the concentration determination method of the present invention on a series of samples (e.g. preparations) (or alternatively termed “reference suspensions”) which contain known concentrations of microorganisms, i.e. samples (suspensions) for which the concentration of microorganisms is or has been determined by an alternative method. Thus, the number of objects corresponding to intact microorganisms may be determined for each of the samples containing different concentrations of microorganisms, and thus the relationship between the number of said objects and a concentration of microorganisms may be established.

A calibration curve is pre-determined, in the sense that it is prepared prior to performing the concentration determination method of the present invention. A calibration curve may, therefore, be prepared separately before determining the concentration of microorganisms in suspension obtained from a given (i.e. every) sample. However, it is preferred that a calibration curve may be prepared and used to determine the concentration of intact microorganisms in multiple suspensions, or put another way, the concentration of intact microorganisms in multiple suspensions may be determined using the same calibration curve. In other words, it is not necessary for the method to comprise the generation of a calibration curve; a pre-prepared calibration curve can be used, and a separate calibration curve does not need to be generated for each sample/suspension. A new or fresh calibration curve may be prepared periodically, e.g. daily, weekly or monthly, or may be prepared batch-wise, e.g. before a new batch of stain is used, and said new calibration curve may be used to determine the concentration of intact microorganisms until it is required that a new calibration curve is to be prepared.

However, a calibration curve that is suitable for determining the concentration of a given microorganism, or type of microorganism, may be provided when performing the methods of the present invention, and it may therefore be preferred that separate calibration curves are prepared for different microorganisms or microorganism types having different characteristics, e.g. different growth patterns. Thus, this need not necessarily be at the level of a particular genus or species of microorganism but may depend, for example, on the morphology and/or growth pattern of the microorganism.

The suitability of a calibration curve for use in determining the concentration of intact microorganisms in a suspension may in some cases depend on the identity of said microorganism, and will determine how accurately the calibration curve allows the concentration of intact microorganisms to be determined from an image analysis value. It may be possible, for example, that a single calibration curve generated using a particular microorganism may be suitable for determining the concentration of a range of different microorganisms, e.g. microorganisms within a single family or genus, and in this way it may only be necessary to prepare a single calibration curve for use in the methods of the present invention. Alternatively, a calibration curve for this purpose may be prepared using imaging data obtained from microorganisms from different families, genera, species or strains, and/or different microorganisms having similar characteristics and/or morphologies, and data obtained therefrom may be combined to provide a single calibration curve.

For example, it may be possible to collect data from different species of non-clustering Gram-negative bacteria, thereby to prepare a calibration curve. A calibration curve thus prepared may therefore be used in determining the concentration of many different (suitable) microorganisms, i.e. microorganisms for which it proves a satisfactory (i.e. representative) correlation between the number of imaged objects and the concentration of microorganisms in a suspension.

Alternatively, if a specific microorganism exhibits irregular or unusual properties, it may be necessary to generate a separate calibration curve for that particular microorganism in order to determine the concentration of that microorganism in a suspension.

A number of different calibration curves, each suitable for use in the determination of the concentration of a different selection of microorganisms, may therefore be provided (i.e. prepared prior to performing the concentration determination method of the present invention). Thus, for example, separate calibration curves may be provided for non-clustering Gram-negative bacteria, non-clustering Gram-positive bacteria, clustering Gram-negative bacteria or yeast. A suitable calibration curve may therefore be selected in order to determine the concentration of a particular microorganism in a sample. Thus, 2, 3, 4, 5 or 6 or more different calibration curves may be prepared, and a suitable calibration curve selected therefrom once imaging of the microorganisms has been performed.

In a preferred embodiment of the invention, information obtained in imaging step (e)(iv) may inform the selection of which calibration curve is to be used in order to determine the concentration of viable microorganisms in a suspension of microorganisms prepared from a particular sample. One or more of the parameters of objects described above (i.e. maximum intensity, modal intensity and/or area or a derived value of the objects as described above) may be determined for the objects detected in step (e)(iv), optionally after background subtraction and/or thresholding steps, and such information may be used to select a suitable calibration curve for that sample. Preferably, a calibration curve is used which is predetermined for clustering or non-clustering microorganisms.

Factors such as the nature of a sample or suspension, the medium in which the microorganisms are resuspended, and the conditions under which the sample and/or suspension is stored or incubated may also all affect the relationship between the concentration of microorganisms in a suspension and the number of objects imaged in step (e)(iv) of the present method, and thus a calibration curve is preferably prepared under similar or the same conditions as those under which a the suspension-stain mixture is imaged.

As noted above, the concentration determination method of the present invention has particular utility in determining the concentration of intact (and therefore viable) microorganisms in a suspension prepared from a sample in the context of performing an AST assay, and in particular in the context of determining the concentration of microorganisms in an inoculum therefor. The present invention therefore provides a method for determining the antimicrobial susceptibility of a microorganism, said method comprising preparing a suspension of microorganisms from a sample and determining the concentration of viable microorganisms in the suspension as outlined above, and performing an AST assay.

Advantageously, the invention provides a method which starts from a clinical sample or clinical sample culture, and which comprises the recovery (or isolation) of viable microorganisms from a clinical sample or clinical sample culture, the determination of the concentration of intact (and hence indicative of viable) microorganisms in a suspension of the recovered microorganisms, and optionally the preparation of an inoculum from the suspension (which may comprise the adjustment of the concentration of microorganisms in the suspension or a portion or aliquot thereof). The suspension of recovered microorganisms or an inoculum prepared therefrom may be used as the inoculum for the AST microbial test cultures which are prepared in the AST assay.

The AST assay may, as described further below, be performed in any convenient or desired way. Accordingly, microbial growth may be assessed (or determined) in the presence of different antimicrobial agents (e.g. antibiotics) and/or amounts or concentrations of antimicrobial agent (e.g. antibiotic). Growth may be assessed directly or by assessing (determining) markers of growth.

Generally speaking, an AST assay is performed by monitoring the effect of an antimicrobial agent on microbial growth. A sample containing microorganisms is used to inoculate culture medium in a series of at least two culture vessels (i.e. to set up at least two AST microbial test cultures), each comprising a different concentration of an antimicrobial agent, and the microorganisms are cultured for a period of time. In this way, a series of at least two different concentrations of an antimicrobial agent is tested in order to determine the amount of agent (e.g. the minimum inhibitory concentration (MIC)) that is required in order to prevent microbial growth. The antimicrobial agent susceptibility value (e.g. MIC value and/or SIR value) obtained thus provides an indication of whether a microorganism is resistant or susceptible to an individual antimicrobial agent.

In addition to inoculating at least two AST microbial test cultures comprising different concentrations of antimicrobial agents, an AST assay will have a positive control condition (culture medium that does not comprise an antimicrobial agent) in order to confirm that the microorganism is viable and is capable of growth in the growth medium provided for the AST assay, and a negative control condition (culture medium which has not been inoculated with a microbial culture and which does not comprise an antimicrobial agent) in order to confirm that the growth medium is not contaminated with a microorganism that is not obtained from the clinical sample. Thus, step (iii) of the method for determining the antimicrobial susceptibility of a microorganism in a sample will generally include setting up suitable positive and negative control conditions, in addition to the at least two different growth conditions.

The positive control sample may be seen in some embodiments as providing a first concentration of an antimicrobial agent (i.e. a concentration of 0 M), and only a second condition comprising an antimicrobial agent may be set up. In such an embodiment, the growth in the positive control condition and the condition comprising an antimicrobial agent may be assessed in order to determine antimicrobial susceptibility. Thus “at least two different growth conditions, wherein . . . each antimicrobial agent is tested at two or more different concentrations” may be seen to encompass an embodiment in which an antimicrobial agent is added to only a single growth condition, and the positive control condition represents a second concentration of the antimicrobial agent.

In a preferred aspect, more than one (i.e. two or more) different antimicrobial agent is tested, thus providing two or more different values for antibiotic susceptibility (e.g. MIC values and/or SIR values), one for each different antimicrobial agent. The combination of different values (e.g. different MIC and/or SIR) values provides the antimicrobial susceptibility profile of a given microorganism, i.e. which of a panel of antimicrobial agents a microorganism is resistant to, and which of a panel of antimicrobial agents a microorganism is susceptible to. Separate positive and negative control conditions may be set up for each separate antimicrobial agent that is tested, if required, however a single positive and a single negative control condition will suffice where multiple different antimicrobial agents are tested.

Microbial growth in the AST method may be assessed by any desired or suitable means, including by any means known in the art. More particularly, microbial growth may be assessed by determining the amount and/or number and/or size of microorganisms and/or microbial colonies or aggregates. As will be discussed in more detail below, in certain preferred embodiments, microbial growth is assessed (determined) by imaging, or alternatively expressed, by visualising the microorganisms. Thus microbial cells, which may include aggregates or clumps (clusters) of cells, or microbial colonies, may be visualised or imaged as a means of determining (or assessing or monitoring) growth. This may include counting of cells or colonies, but is not limited to such methods and includes any means of visually assessing the amount of microbial growth by assessing (or determining) the size, area, shape, morphology and/or number of microbial cells, colonies or aggregates (the term “aggregate” includes any collection of cells in physical proximity e.g. a clump or cluster; this may include non-clonal clumps/clusters of cells which have aggregated or stuck together (e.g. neighbouring cells which have become aggregated) as well as clonal colonies).

The parameter used to measure microbial growth may, but need not, vary according to the identity of the microbe and the antimicrobial agents used. Indeed, depending on the organism and the antimicrobial agents used, the morphology or growth pattern of the cells may be affected, and this may be altered or changed from the “normal” or “typical” morphology or growth pattern, e.g. in the absence of the antimicrobial agent. Whilst some AST growth monitoring methods may depend on detecting such changes, it is not essential according to the present invention to take such changes into account and the amount (e.g. area) of microbial growth or biomass may be determined irrespective of morphology and/or growth pattern. Thus the same growth monitoring method may be used regardless of the microbial cell and/or antimicrobial agents used. Methods for performing the AST assay are described further below.

The present invention provides a method of determining the concentration of intact, or viable, microorganisms in a suspension, and this information can be used to accurately provide a particular concentration of microbial cells in the test microbial cultures. The concentration of microorganisms in at least a portion of the suspension may be adjusted once the concentration has been determined, in order to provide an inoculum for inoculating the test microbial cultures in step (iii). As discussed above, however, this does not preclude an additional preliminary adjustment before the concentration has been determined. Thus, the concentration of microbial cells in the suspension may optionally, or if necessary, be adjusted, e.g. to fall within a range suitable for use in an AST assay. This adjustment may not be required in every instance, i.e. the suspension may be used directly to inoculate the series of test microbial cultures that are set up in step (iii) (i.e. the suspension may be used directly, i.e. without any further adjustment). Alternatively, the suspension (or an aliquot thereof) maybe adjusted to a desired or pre-determined concentration. Still further alternatively the suspension may be used directly (i.e. without adjustment) to inoculate the series of test microbial cultures, and the concentration of microorganisms in the test microbial cultures may be adjusted, if necessary, to a desired or pre-determined concentration. Any such adjustment will be based on the concentration of viable microorganisms determined in the concentration determination method (i.e. based on the concentration of microorganisms in the suspension).

Thus, the methods of the present invention may further comprise a step (f) in which the concentration of microbial cells in the suspension, or a portion thereof, and/or in a test microbial culture, is adjusted. More particularly the concentration may be adjusted to increase or to decrease the number, or concentration, of microbial cells. Such an adjustment may be made in the context of an AST assay, as discussed above, but may also be made in any other context for any desired reason, e.g. to aliquot the recovered microorganisms for further analysis (e.g. genetic analysis), storage (e.g. freezing), etc.

As discussed above, in certain embodiments the methods may comprise an initial adjustment, preferably an initial dilution, before the concentration of microorganisms in the suspension is determined. This may be viewed as part of the adjustment step (e.g. as an initial or preliminary or adjustment). Alternatively, this may be viewed as a separate initial (preliminary, or blind) adjustment which is performed independently of any adjustment step performed after the concentration has been determined. Once the concentration of microorganisms in the suspension is determined, further adjustment of the concentration of microorganisms may be performed (e.g. to fall within a range suitable for use in an AST assay), if required, in view of the concentration of microorganisms that is determined in step (e)(v) of the present invention. Thus, in an embodiment, the methods may comprise an additional step (f) of adjusting the concentration of microorganisms in at least a portion of the suspension, after the concentration has been determined in step (e). In another embodiment, the methods may comprise performing an initial adjustment of the concentration of microorganisms in at least a portion of the suspension before the concentration has been determined, and then performing a further adjustment of the adjusted suspension or portion thereof, after the concentration has been determined in step (e). In an embodiment, step (f) may be viewed as a step of performing such a further adjustment. Beneficially, performing such an initial adjustment (e.g. in the course of adjusting the concentration of microbial cells in the suspension) may reduce the time required to prepare a suspension (e.g. an inoculum) having a desired concentration of microorganisms once the concentration of microorganisms in a suspension has been determined in step (e)(v).

Accordingly, in one embodiment, the microorganism concentration is adjusted in at least a portion of the suspension. The at least portion of the suspension in which the microorganism concentration is adjusted preferably is a portion of the suspension obtained in step (d) which was not stained in step (e)(iii), i.e. it is an unstained portion of the suspension.

Adjustment of the concentration of at least a portion of the suspension may provide an inoculum for inoculating the test microbial cultures in step (iii). Thus for example the concentration of microbial cells in the inoculum may be increased e.g. by culturing the sample for a period of time to allow the microbial cells to grow, or decreased e.g. by dilution prior to inoculating the test microbial cultures, or in the course of inoculating the test microbial cultures e.g. by selecting an appropriate amount (e.g. volume) to be used to set up the test cultures, either by adding to solid (e.g. dried antimicrobial agent, such as freeze-dried or vacuum-dried antibiotics) or by dilution when a portion or aliquot of the inoculum is added to a volume of antibiotic and/or culture medium for the AST test. Accordingly the test microbial cultures may be inoculated with the suspension (or aliquot thereof) or with an adjusted (e.g. diluted) inoculum therefrom.

In one embodiment, wherein the suspension comprises a microbial concentration that is higher than desired, e.g. a microbial concentration which is too high to be used in an AST assay, the microbial culture is diluted using an appropriate buffer or culture medium (e.g. liquid culture medium) in order to reduce the cell density to a suitable level, e.g. a suitable level for an AST to be performed. In the case of an AST assay, the dilution is preferably performed using the culture medium which is to be used to perform the AST assay. In one embodiment this may be performed using a Muller Hinton (MH) broth. Adjusting the concentration may, for example, comprise a dilution based on the concentration determined in step (ii) of the AST method.

In an alternative embodiment, wherein the suspension comprises a microbial concentration that is too low to be used in an AST assay, the suspension may be cultured (or further cultured) for a period of time in order to allow the microorganisms present therein to grow and increase in number. The concentration of microbial cells present in the suspension may be monitored either continuously or at a series of individual time points until the concentration of microorganisms reaches a sufficiently high cell density that an AST assay may be performed. Growth of the microbial culture at this stage may be monitored by any of the methods described herein for monitoring growth in the AST assay itself, e.g. imaging or counting of cells or colonies, and/or the concentration determination method of the present invention may be performed following a period of growth.

Thus, in one embodiment the present invention utilises an inoculum (e.g. suspension or diluted suspension) having a standard microbial concentration (e.g. 0.5 McFarland units or 10⁸ CFU/ml), or a concentration in the region thereof, in order to inoculate the test cultures used in an AST assay. The concentration of microbial cells present in the suspension may optionally, or if necessary be adjusted, that is increased or decreased depending on the number of cells present in the sample, in order to obtain a suspension having a standard concentration. Alternatively, the concentration of microbial cells present in the suspension may lie within a standard range, without the need for an adjustment step to be performed. Regardless, the concentration of microbial cells present in the suspension is determined by the method of the present invention, and may be adjusted as and if required to obtain a suspension having a standard concentration. Alternatively, the suspension may be used without adjustment and the concentration of microbial cells in the test microbial cultures may be adjusted (e.g. by selecting an appropriate dilution factor for setting up the test culture or an appropriate volume), based on the concentration of microorganisms determined in the suspension.

AST assays typically utilise microbial cultures having set (or standard or standardised) cell densities or microbial concentrations in order to allow results obtained from one sample or in one location to be compared with those obtained elsewhere, as the response of microorganisms to antimicrobial agents is known to vary with the concentration of microorganisms in a sample, as well as the type and concentration of the antimicrobial agent itself. Factors influencing clinical outcomes such as the dosage of an antimicrobial agent and the treatment regime prescribed to a patient are based on results obtained from AST assays performed according to set standard criteria.

The results obtained in an AST assay performed using a ‘non-standard’ (or “non-standardised”) microbial culture (the antimicrobial susceptibility profile of a microorganism, or a set of MIC and/or SIR values and/or any other values indicative of antimicrobial susceptibility) may differ from the results obtained in an AST assay performed according to standard criteria, e.g. using a ‘standard’ microbial culture. However, the degree to which a antimicrobial susceptibility value obtained using a non-standard microbial culture varies from a antimicrobial susceptibility value obtained using a standard microbial culture may be determined, if the concentration of microbial cells in the suspension or inoculum used to inoculate the AST test cultures is known. It is thereby possible to calculate a theoretical ‘standard’ antimicrobial susceptibility value (e.g. MIC and/or SIR value) from a antimicrobial susceptibility value obtained using a non-standard microbial culture.

The degree to which the susceptibility value obtained using a non-standard microbial culture varies from a ‘standard’ MIC value may vary depending on the nature of the microorganism and the antimicrobial agent, and can be determined separately, e.g. for each different antimicrobial agent that is tested and for microbial cultures comprising different concentrations of microbial cells.

The present invention thus provides a method to determine the antimicrobial susceptibility profile of a microorganism using an inoculum comprising a non-standard concentration of microbial cells, wherein the concentration of microbial cells in the test microbial cultures is measured (indirectly, by measuring the concentration of microbial cells in the suspension used to inoculate said test microbial cultures or to prepare the inoculum) before the AST assay is performed (i.e. the concentration of microbial cells in the suspension is determined, and the susceptibility value (e.g. MIC and/or SIR value) obtained in the AST assay may be adjusted based on the concentration of microbial cells in the test microbial cultures prepared therefrom to give a standard value (e.g. MIC and/or SIR value.

As described above, the standard inoculum used to set up an AST test assay in the methods of the prior art typically is approximately 0.5 McFarland units. As mentioned above, this corresponds to approximately 10⁸ CFU/ml. This is typically diluted in a 1:200 dilution to provide test microbial cultures comprising approximately 5×10⁵ CFU/ml. However, whilst the methods of the present invention may use these standard values, and it is generally preferred for the concentration of microorganisms in the inoculated microbial test cultures in the AST test to be in the range of 4.5×10⁵±80% or 5×10⁵±60%, it is possible in the methods of the present invention for the inoculum (e.g. the suspension and the inoculum prepared therefrom) and/or the test microbial cultures to comprise any defined or pre-determined concentration of microbial cells, provided the concentration of microbial cells in the test microbial cultures that are used to obtain an AST value is known. Thus, in other embodiments the concentration of microorganisms in the inoculated microbial test cultures in the AST test may be in the range of 1×10⁵±80% or 5×10⁴±80%, or 5×10⁴±60%, etc.

The concentration of microbial cells in the suspension may therefore be any desired or pre-determined concentration that is suitable for setting up a microbial test culture in an AST method. It may therefore be at least 10, 10², 10³, 10⁴, 10⁵, 10⁶, 10⁷, 10⁸, 10⁹, 10¹⁰, 5×10¹⁰ or 10¹¹ CFU/ml. Preferably the concentration of microbial cells in the suspension will be 10-10¹¹, 10²-10¹¹, 10³-10¹¹, 10⁴-10¹¹ CFU/ml, 10⁵-10¹¹ CFU/ml, 10⁶-10¹¹ CFU/ml, 10⁷-10¹¹ CFU/ml, 5×10⁶-10¹¹ CFU/ml, 2×10⁶-10¹¹ CFU/ml, 10⁶-10¹¹ CFU/ml, 5×10⁶-5×10¹⁰ CFU/ml, 2×10⁶-5×10¹⁰ CFU/ml, or 10⁶-5×10¹⁰ CFU/ml.

The statistical reliability of an AST assay performed using an inoculum having a low concentration of microorganisms may be worse than in embodiments where the inoculum contains a higher concentration of microorganisms. Thus, in certain embodiments, if a particularly low concentration of microorganisms is determined in the suspension, it may be desirable or advantageous not to continue with the AST assay at that stage. Thus, in certain embodiments, where the concentration of microorganisms in the suspension is below 1×10³ CFU/ml, or more preferably below 1×10⁴, 1×10⁵ or 1×10⁶ CFU/ml, the AST assay may not be performed with the suspension (i.e. the AST method is not performed beyond step (ii)). Optionally, the concentration of microorganisms in the suspension may be allowed to increase before the concentration determination method is repeated (e.g. following a period of culture), and if the suspension contains a sufficiently high concentration of microorganisms at this later stage, it may be possible to then proceed with the AST assay.

The AST method of the invention, which allows non-standard concentrations to be used in the AST test, has particular utility if the concentration of microbial cells in the suspension is below the standard concentration, as it may bypass the need to incubate said suspension for a period of time in order to allow the concentration of microbial cells in the suspension to increase, e.g. to a level above that of the standard concentration.

The AST method presented herein may be viewed as a method to determine the ‘standard’ antimicrobial susceptibility profile of a microorganism by adjusting the susceptibility (e.g. MIC and/or SIR) values obtained by performing an AST assay using a non-standard microbial culture. Viewed another way, this provides a theoretical way to adjust the concentration of microbial cells that is used to inoculate the test cultures used in an AST assay, thereby to calculate the antimicrobial susceptibility of a microorganism.

Whilst it is possible to use a non-standard sample to inoculate the test cultures used in the present invention, in an alternative embodiment the present invention provides methods to physically adjust the concentration of microbial cells present in a suspension and/or test microbial cultures so that the concentration of microbial cells in the test microbial cultures corresponds to a standard or standardised concentration, (e.g. about 5×10⁵ CFU/ml) in order that a standard AST assay may be performed.

The suspension, or an inoculum prepared therefrom, is used to inoculate the test microbial cultures. As discussed above, the suspension may be added to culture medium, i.e. the suspension may be diluted, or diluted further, at the stage of setting up the test microbial cultures (step (iii) of the AST method). Thus, the test microbial cultures may be adjusted at this point to comprise any desired or pre-determined concentration. Thus, the test microbial cultures will comprise an initial concentration of microbial cells of at least 10, 10¹, 10², 10³, 10⁴, 10⁵, 10⁶, 10⁷, 10⁸ or 10⁹ CFU/ml, preferably 10²-10⁸, 10³-10⁷ or 10⁴-10⁶ CFU/ml. As noted above, the test microbial cultures may, therefore, be set up to a final concentration of 5×10⁴±80%, 1×10⁴±80%, 4×10⁵±80%, 4.5×10⁵±80% or 5×10⁵±80%.

It is noted however that what constitutes a ‘standard’ sample may vary depending on the identity of the microorganism, i.e. the concentration of microbial cells present in the suspension may depend on the identity of the microorganism. Preferably the concentration of microbial cells in the suspension will be 10-10¹¹, 10-10¹⁰, 10-10⁹, 10²-10⁹, 10³-10⁹, 10⁴-10⁹ CFU/ml, 10⁵-10⁹ CFU/ml, 10⁶-10⁹ CFU/ml, 10⁷-10⁹ CFU/ml.

Recognised and prescribed conditions for AST assaying exist, and may be followed in order that readily comparable results may be obtained which are comparable to, or may be compared with, tests performed in other laboratories.

This may involve for example the use of a prescribed medium and culture conditions. In certain embodiments, medium for microbial culture may be a liquid medium, i.e. the culture medium may be a liquid.

In certain embodiments it may be advantageous or desirable to set up test microbial cultures in parallel having different media for the growth of different microorganisms. This may be useful, for example, if the identity of the microorganisms in the sample (and hence suspension) is not known, or if their growth patterns or requirements have not been fully characterised. Thus for example, parallel microbial test cultures in the AST method may be set up which contain, or do not contain fastidious supplements in the growth medium, or in other words, parallel test microbial cultures in fastidious or non-fastidious media. Fastidious media are well known in the art and both pre-prepared fastidious media and fastidious supplements are widely and commercially available. Fastidious supplements included for example lysed blood preparations (e.g. lysed horse blood), serum, various vitamins and/or minerals, cofactors, etc., e.g. beta-nicotinamide. Conveniently, fastidious supplements may be added to culture media as part of a dilution protocol. Further, whether or not fastidious media or supplements are used may depend on the concentration of microbial cells which is determined for the suspension. For example, if the concentration is low, e.g. if there is less than 2×10⁶ CFU/ml microbial cells in the suspension, the use of microbial test cultures with fastidious media/supplements may be omitted from the AST method.

In certain embodiments, it may also be advantageous to set up test microbial cultures in parallel having different media optimised for testing susceptibility to particular antimicrobial agents. Additives necessary for specific antibiotics may be included in test microbial cultures. For example, polysorbate 80 may be included, and/or an increased calcium concentration may be provided in certain test microbial cultures.

Microorganisms may be grown in the presence of a variety of antimicrobial agents to determine their susceptibility to a given antimicrobial agent. The antimicrobial agents may be selected based on the identity of the microorganism, if known, and preferably also on the nature of any genetic antimicrobial resistance markers identified within the microorganism. The antimicrobial agents, and the amounts to be used, may also be selected according to current clinical practice, e.g. according to which antimicrobial agents are currently used in practice to treat the identified microorganism, in order that the susceptibility of the microorganism to the currently accepted or recognised antimicrobial treatment of choice can be assessed.

Thus antimicrobial agents can be selected based on those known to be effective against the identified microorganism, or those currently used in practice to treat the microorganism, and excluding any agents to which resistance might be expected based on the presence of resistance markers, or such agents might be included and the amounts used might be selected to allow the determination of an amount or concentration of the antimicrobial agent that may be effective, despite the presence of the resistance marker. Antimicrobial agents are added to culture medium to a range of final concentrations or amounts. In one embodiment of the present invention a dilution of the antimicrobial agent may be performed. In a preferred format of the invention antimicrobial agents in pre-determined amounts, to yield pre-determined concentrations after being dissolved, are pre-deposited in wells where culture media with microorganisms are added before the AST. The pre-deposited antimicrobial agents are preferably dried, e.g. freeze-dried or vacuum-dried, formulations.

The step of growing, or culturing, the suspension/microorganisms therefrom in the AST assay may take place by any known or convenient means. Solid or liquid phase cultures may be used.

Thus for example, in one preferred embodiment, the microorganisms may be cultured on or in a plate or other solid medium, or in a vessel (e.g. a well of a plate) containing a liquid medium, containing the antimicrobial agent and microbial growth may be determined by visualising (e.g. imaging) the microorganisms (i.e. imaging the plate etc.) Thus, the culture is visualised or imaged directly as a means of monitoring or assessing growth. Accordingly in one preferred embodiment the cultures are analysed directly to monitor/assess growth. For example, the cultures may be grown in the wells of a plate, or compartments of a carrier substrate and the wells/compartments may be imaged.

Alternatively, samples (or aliquots) may be removed (or taken) from the AST test cultures, at intervals, or at different time points and the removed samples (aliquots) may be analysed for microbial growth. This may be done by any means, including for example by means of molecular tests, e.g. nucleic acid based tests, Thus detection probes and/or primers may be used which bind to the microbial cells or to components released or separated from microbial cells. This may include for example nucleic acid probes or primers which may hybridise to microbial DNA. In other embodiments, microbial cells may be detected directly, e.g. by staining, as described in more detail below.

Each antimicrobial agent may be used at at least two concentrations, in addition to a positive control in which the microorganism is allowed to grow in the absence of any antimicrobial agent as well as at least one negative control that are cultured in absence of added test aliquot. For example, 2, 3, 4, 5, 6, 7, or 8 or more concentrations of an antimicrobial agent are used. The concentrations used in a dilution series may differ two-fold between respective concentrations.

The term antimicrobial agent includes any agent that kills microorganisms or inhibits their growth. Antimicrobial agents of the present invention may particularly include antibiotics and antifungals. Antimicrobial agents may be microbicidal or microbiostatic. Various different classes of antibiotic are known, including antibiotics active against fungi, or particularly groups of fungi and any or all of these may be used. Antibiotics may include beta-lactam antibiotics, cephalosporins, polymyxins, rifamycins, lipiarmycins, quinolones, sulphonamides, macrolides, lincosamides, tetracyclines, aminoglycosides, glycopeptides, cyclic lipopeptides, glycylcyclines, oxazolidinones, lipiarmycins or carbapenems. Preferred antifungals of the present invention may include polyenes, imidazoles, triazoles and thiazoles, allylamines or echinocandins. Antimicrobial agents are continuously being developed and it is understood that it will also be possible to analyse future antimicrobials with the current invention.

Preferably, at least one of the test microbial cultures comprises fastidious medium. More preferably, at least two of the test microbial cultures, e.g. at least two different growth conditions comprising the same antimicrobial agent at a different concentration, may comprise fastidious medium, such that the antimicrobial susceptibility of a microorganism to a particular antimicrobial agent under fastidious growth conditions.

Antimicrobial susceptibility may be determined by culturing the microorganisms from the suspension, and analysing the AST cultures over a range of time points.

The AST cultures may be analysed at multiple time points to monitor microbial growth. For example, cultures may be analysed at time points 0, 1, 2, 3, 4, 5, 6, 7, 8, 9, 10, 11, 12, 13, 14, 15, 16, 17, 18, 19, 20, 21, 22, 23 or 24 hours after the initiation of culture. A culture may be analysed immediately after the initiation of culture, where t=0. Cultures may also be analysed at time periods beyond 24 hours after the initiation of culture. Typically cultures might be analysed at 0, 1, 2, 3, 4, 6 and 24 hours after the initiation of culture. However, results obtained using the method show that short incubation times can be sufficient for detecting differential microbial growth e.g. 4 hours. Accordingly, shorter total incubation time of up to 8, 7, 6, 5, 4, 3 or 2 hours may also be used, e.g. analysing every hour or every 2 hours or 90 minutes. As noted above, cultures are generally analysed at two or more time points, e.g. at two or more time points up to 4, 5 or 6 hours of culture. In certain embodiments, the AST cultures may be analysed at more frequent time points. A culture may be analysed at t=0, and may subsequently be analysed at intervals of 1, 2, 3, 4, 5, 10, 15, 20, 25 or 30 minutes. Accordingly, the total incubation time required when such short analysis intervals are used may also advantageously be reduced, and thus a shorter incubation time of up to 10, 15, 20, 25, 30 or 60 minutes may be used.

The monitoring or assessing of microbial growth in the AST assay may take place by monitoring growth continuously or at intervals over a time period (e.g. up to 10, 15, 20, 25 or 30 minutes or up to 1, 2, 3, 4, 5, 6, 7 or 8 hours), or by comparing the amount of microbial cell matter at the time the AST growth culture (test microbial culture) is initiated (t0) with the amount of microbial cell matter at a later time point (e.g. at up to 10, 15, 20, 25 or 30 minutes or up to 1, 2, 3, 4, 5, 6, 7, or 8 hours), i.e. the growth that has taken place in the intervening time. Alternatively, the amount of microbial cell matter may be determined at two or more different time points (e.g. measuring the first time point after 1, 2, 3, 4, 5, 10, 15, 20, 25 or 30 minutes or 1, 2, 3 or 4 hours, and measuring a second time point 1, 2, 3, 4, 5, 10, 15, 20, 25 or 30 minutes or 1, 2, 3, 4, 5, 6 or 7 hours after the first time point, or 2, 3, 4, 5, 6, 7, 8, 9, 10, 15, 20, 25, 30, 35, 40, 45, 50 or 55 minutes, or 2, 3, 4, 5, 6, 7 or 8 hours after the initiation of culture) and the amount of growth may thereby be determined. In preferred embodiments, the degree of microbial growth may be determined at more than one time point, i.e. at at least two time points.

In another embodiment, growth is assessed in a test microbial culture grown in the presence of an antimicrobial agent with a test microbial culture grown in the absence of antibiotics (e.g. a positive control) at only one time point, e.g. at 1, 2, 3, 4, 5, 6, 7 or 8 hours. Monitoring growth at a time point (or two or more time points) after the initiation of the AST growth culture may advantageously allow a more accurate result to be achieved by avoiding measuring growth during the lag phase of microbial growth, as any differences between microbial growth under different conditions during this period of time will be small and difficult to detect. A first measurement may be taken according this method after 30 minutes or 1, 2, 3 or 4 hours, and a second measurement may be taken 1, 2, 3, 4, 5, 6, 7 or 8 hours after the first time point).

It will be apparent, however, that for certain microorganisms, e.g. certain anaerobes, mycobacteria or fungi, microbial growth may be less rapid, and thus an AST assay may need to be performed for a longer period of time. Thus, according to certain embodiments of the present invention, it may be necessary or desirable to perform the AST assay by measuring microbial growth for 8, 9, 10, 11 or 12 hours or more, e.g. 12, 18 or 24 hours. Suitable measurements at one or more time points may be taken accordingly.

In a preferred embodiment, growth may be measured in at least two growth conditions (e.g. each growth condition), relative to the initial number (amount or concentration) of microbial cells in each growth condition.

Culture of the test microbial cultures may take place at any temperature that promotes microbial growth, e.g. between about 20° C. and 40° C., or 20 to 37° C., preferably between about 25° C. and 37° C., more preferably between about 30° C. and 37° C. or 30 to 35° C. In one embodiment the AST cultures may be cultured at about 35° C.

Many methods for monitoring or assessing microbial growth are known and are used in AST assays, for example including turbimetric measurement, colorimetric determination, light detection, light scattering, pH measurement, spectroscopic measurements, fluorometric detection measuring of degradation products of antibiotics or microbial, measuring nucleic acid content or measuring production of gas, e.g. CO₂. Any of these may be used. However, according to a preferred embodiment of the present invention growth may be detected and assessed by determining or assessing the number and/or amount and/or size and/or area of microbial cells by imaging methods, As noted above, the microbial cells can include cells in colonies and/or aggregates. This may be achieved by assessing or determining the number or amount of microorganisms present before and/or after growth in presence of antimicrobial agents by any of the methods known to measure or detect microorganisms. Such a determination may involve determining the number and/or size of microbial cells, aggregates and/or colonies. Again, techniques for this are known and available. Thus, growth may be measured by monitoring the number and/or amount and/or size of microorganisms and/or microbial cells and/or colonies and/or aggregates over time. This may be measured directly or indirectly. The number or amount of microorganisms may be measured directly by haemocytometry, flow cytometry, or automated microscopy. Microorganisms may be fixed and/or permeabilised prior to detection. Alternatively, microorganisms may be detected under in vivo conditions.

Methods for AST assaying by bacterial cell count monitoring using flow cytometry are described in Broeren et al., 2013, Clin. Microbiol. Infect. 19. 286-291. Methods for performing AST assays in which bacteria are grown and enumerated by automated microscopy in multi-channel fluidic cassettes are described by Price et al., 2014, J. Microbiol. Met. 98, 50-58 and by Metzger et al., 2014. J. Microbiol. Met. 79, 160-165, and by Accelerate Diagnostics (see for example WO 2014/040088 A1, US 2014/0278136 A1 and U.S. Pat. No. 8,460,887 B2). In these methods, bacteria are immobilised and grown on a surface, and individual bacteria and/or colonies are assessed for viability and/or growth (including measuring colony growth) by imaging the surface at two or more time points. Such methods may be used according to the present invention. Other methods known are as described by Fredborg et al., J Clin Microbiol. 2013, 51(7): 2047-53, and by Unisensor (U.S. Pat. No. 8,780,181) where bacteria are imaged in solution using bright-field microscopy by taking a series of stacked images (object planes) of the solution, and counting the bacteria present in the sample.

Any of the methods based on using imaging to monitor microbial growth described herein or known in the art may be used in the AST step of any method disclosed herein for determining AST (step (iv) of the AST method set out above). However, in certain embodiments, the microbial growth determination step in the AST methods (i.e. step (iv) of assessing the degree of microbial growth) in the AST method does not rely on counting individual cells or on monitoring the growth of individual cells or colonies (e.g. on monitoring an increase in size of an individual cell or colony e.g. according to the methods of Accelerate Diagnostics Inc.). Thus, the presently disclosed methods are not limited to (and in certain embodiments dos not involve) using a fixed position for imaging an AST culture or AST culture sample. Rather, it is preferred to monitor the bulk growth of cells in the AST culture, e.g. by imaging bulk cells in the field of view. The amount (e.g. area) of microbial cell matter (biomass) in the field of view may be determined by imaging. The cells/microbial biomass may be detected directly (e.g. by the microscope or camera etc.) e.g. using bright field microscopy or the microbial cells may be stained for detection, e.g. by adding stain to the AST culture or culture sample after the predetermined or required time period of growth. However, in other methods individual cells may be counted, or the growth of an individual cell or colony can be monitored. Thus, other methods than those specifically described and demonstrated herein may be used to determine or assess microbial growth in AST test cultures, and the methods disclosed herein for preparing microbial suspensions and/or determining the microorganism concentration therein may have utility in other AST methods.

Thus in the step (iv) of assessing the growth in the microbial test cultures, this is preferably done by imaging the test cultures over a large (significant or substantial) part of the culture available for imaging. Furthermore, in step (iv) the imaging may be done without pre-selecting a population or part of the test culture for imaging. Time-lapsed images of the liquid (broth) culture may be generated.

In a further particular embodiment, the AST cultures may be imaged or visualised directly without immobilising the microbial cells or without driving or actively transporting them to a surface, e.g. without applying a force, such as electrophoresis, to localise the cells to a detection location or surface for imaging.

In such imaging methods, algorithms may be applied to determine a value for the amount of microbial growth from the images according to methods and principles well known in the art. Thus, statistical methods may be applied to the images of microbial cells, based on the number, size, and/or area of microbial cell matter/biomass in the images (e.g. the amount of all the microbial cell matter in the image/field of view, for example total cell matter imaged). Algorithms may be written to take account of different growth patterns and/or morphologies, based on the identity of the microorganism and the antimicrobial agent present in the culture. An exemplary image analysis algorithm for use in measuring the amount of microbial biomass in a sample, and hence microbial growth, which combines thresholding and texture filtering, is described in co-pending application WO 2017/216312, and such methods may be used to assess microbial growth in the AST methods of the present invention. Such counting or imaging methods allow a digital phenotypic analysis of the microorganism in the AST assay. Data has been obtained which shows that such digital phenotypic determinations deliver a MIC value similar to that of reference techniques (e.g. microbroth dilution).

A particular advantage of using such methods is that antimicrobial susceptibility testing may be performed on test microbial cultures comprising a wide range of concentrations or amounts of microorganisms, and it is not necessary to use a standardised microbial titre prior to perform the antimicrobial susceptibility testing. A useful feature of the present invention is the ability to use different concentrations of microorganisms. A test microbial culture or sample comprising at least 10³CFU/ml may be used in the methods of the invention, for example samples (AST test samples) comprising at least 10³, 10⁴, 10⁵, 10⁶, 10⁷, 10⁸, 10⁹, 10¹⁰ or 10¹¹ CFU/ml may be used. Alternatively a test microbial culture or sample comprising less than 10³ CFU/ml may be used, for example at least 10² CFU/ml. A test microbial culture or sample comprising less than 10² CFU/ml may also be used in the methods of the present invention.

Although bright field imaging represents one format for assaying the concentration of microbial cells in a test microbial culture, in one embodiment of the present invention, microorganisms may be detected by adding a marker that stains microorganisms (i.e. a stain or dye) prior to determining the number or amount of microorganisms the AST test cultures or by methods which utilize an intrinsic property of the microorganism such as e.g. phase contrast or any other method known in the art for quantifying the number of bacteria in the sample. Suitable stains might include coloured or fluorescent dyes, for example Gram staining or other staining for peptidoglycan or DNA staining, as a means of visualising the microorganism. In one particular embodiment of the present invention, DNA within a microorganism may be stained using Vybrant® DyeCycle™. Other DNA stains are well known and available. Indeed the number of stains available in the art for staining bacteria is vast and large numbers of such stains have been documented, including in standard reference texts, and are commercially available, e.g. from Life Technologies. Direct labelling of microorganisms by staining is easy to perform, convenient and cost-effective, and therefore represents a preferred embodiment.

Thus for example, the microorganisms may be grown for the AST assay in wells of a microtiter plate (i.e. each test microbial culture may be in a well of a plate), and the end of the growth periods the dye or stain may be added and the plate wells may be imaged and the number or amount of microorganisms or microbial cell matter may be assessed, by determining the number and/or size of microbial cells, aggregates or colonies e.g. by counting or imaging. Alternatively, microorganisms may be enumerated using a flow cytometer or similar type of instrument, for example the Aquila 400 instrument from Q-linea AB (Sweden), e.g. as described in U.S. patent Ser. No. 10/112,194.

Algorithms for image analysis are well known and available in the art, to be able to analyse the image and derive or obtain a value for the amount of microbial biomass etc. As mentioned above, one such image analysis technique is described in WO 2017/216312 and this represents a preferred means of assessing and determining microbial growth in the AST test.

Further algorithms may be used to derive an antimicrobial susceptibility value (e.g. a MIC and/or SIR value) for one or more antibiotics for the microorganism in the sample. In this regard, whilst an identification of the microorganism may assist in setting up the AST test, it is not a prerequisite of the method and microbial ID does not need to be known when the method is performed or set up. Thus, in terms of speed of test result, the AST method may be started when the identity (ID) of the microorganisms in the sample is unknown, but the ID may be used in the interpretation of the results, for example when the AST microbial test cultures are imaged, and/or when the results of the imaging are analysed. An antimicrobial susceptibility value (e.g. a MIC value) may be obtained without microbial ID, but ID information is important in determining or interpreting SIR (susceptible/intermediate/resistant) information on the microorganism. Data processing techniques to derive or obtain MIC and/or SIR information from the growth data obtained from the imaging analysis are well known and available to the person skilled in the art.

In an alternative embodiment a microorganism may be specifically labelled via a biological feature within or on the microorganism. A “biological feature” may for example be a molecule in or on the microorganism e.g. a protein or other biomolecule expressed or located on the cell surface. For example a label, e.g. a coloured or fluorescent label, may be coupled to a protein or other affinity binding molecule that binds specifically to a particular biological feature. In one embodiment the protein may be a lectin, affibody or antibody, or antibody fragment. The microorganisms labelled in this way may be detected e.g. enumerated as previously described.

In a further embodiment proximity probes may be used to detect a specific biological feature within or on a microorganism.

In a further alternative embodiment of the present invention the microorganisms in the test microbial cultures may be detected and enumerated using a padlock probe and RCA-based amplified single molecule detection (ASMD) method. Such methods enable single microbial cells to be detected and counted. Thus, the microorganism may be detected by binding of the padlock probe and the number of microorganisms may be measured indirectly by an amplified signal generated via RCA of the circularised padlock probe. Each RCA product (blob) may be indicative of a single microorganism. Microorganisms may be lysed and padlock probes may be used which are designed to hybridise to one or more nucleotide sequences of the microorganisms. This may include a step of separating DNA, and preferably of selectively separating, or enriching for, microbial DNA. Since in the AST assay the test microbial cultures are usually less complex than in initial sample, a simplified protocol for separating or enriching microbial DNA may be used, involving for example filtration to separate microorganisms and microbial cell lysis or simply direct microbial cell lysis.

Alternatively, affinity binding molecules may be used which bind to one or more molecules present on a microorganism or within a lysed microorganism, such an affinity probe being provided with an nucleic acid label or tag to which a padlock probe may hybridise i.e. akin to an immunoRCA detection procedure. Similarly proximity probes may be used to bind to a target in or on a microorganism and the nucleic acid domains of the proximity probes may be used to template the ligation of a padlock probe and optionally also prime its amplification by RCA. Procedures for this are widely known and described in the literature. Circle-to-circle amplification (C2CA) as described for example in in Dahl et al, 2004, PNAS USA, 101, 4548-4553 and WO 03/012199 Dahl et al, 2004, PNAS USA, 101, 4548-4553 and WO 03/012199 may be used for signal amplification. The number of microorganisms in a sample can therefore be estimated by counting the number of blobs, which may be labelled e.g. fluorescently-labelled as described above ‘blobs’ within a sample. This thus provides another convenient means of obtaining a digital phenotypic susceptibility readout.

It is generally speaking advantageous in performing an AST assay for the microbial culture under test to be pure, i.e. for there to be a single microorganism. However, this is not an essential feature, and it is possible to use microbial detection methods based on visualisation or imaging to perform AST assays, for example methods as provided by Accelerate Diagnostics which use imaging of bacteria on a surface and not in solution, or indeed methods in which labelled microorganisms are detected in fluidic systems e.g. the automated microscopy fluidic cassette-based systems of Price et al., 2014, J. Microbiol. Met. 98, 50-58 and by Metzger et al., 2014. J. Microbiol. Met. 79, 160-165, discussed above. Any cell-by-cell detection, or shape recognition and/or identification methods may be used for AST assaying of samples which contain more than one microorganism. It is further known that different microorganisms may be affected differently by the same antibiotic and therefore the appearance of an organism upon treatment with a specific antibiotic may be used for identification and AST determination for each microorganism in co-cultures.

Conveniently the methods of the invention may be automated. Any one of more of the steps may be automated, preferably any or all of steps (a) to (e). Various specific or preferred steps discussed above lend themselves well to automation, for example contacting an aliquot with the stain and/or diluting an aliquot of the suspension, and/or imaging an aliquot/stain mixture in the concentration determination methods of the present invention, as well as AST assaying and recovery of microorganisms from a sample. Automatic culturing methods have already been developed, including for blood culture methods, and these can be combined, for example, with automated concentration determination and/or AST assaying for use according to the present invention. Automation would provide the advantage of speed and ease of operation, as well as multiplexing ability, which are of importance in clinical laboratory setting and especially important in the diagnosis of sepsis.

The methods of the invention and/or as disclosed herein will now be described in more detail in the Examples below with reference to the following figures.

In the Figures,

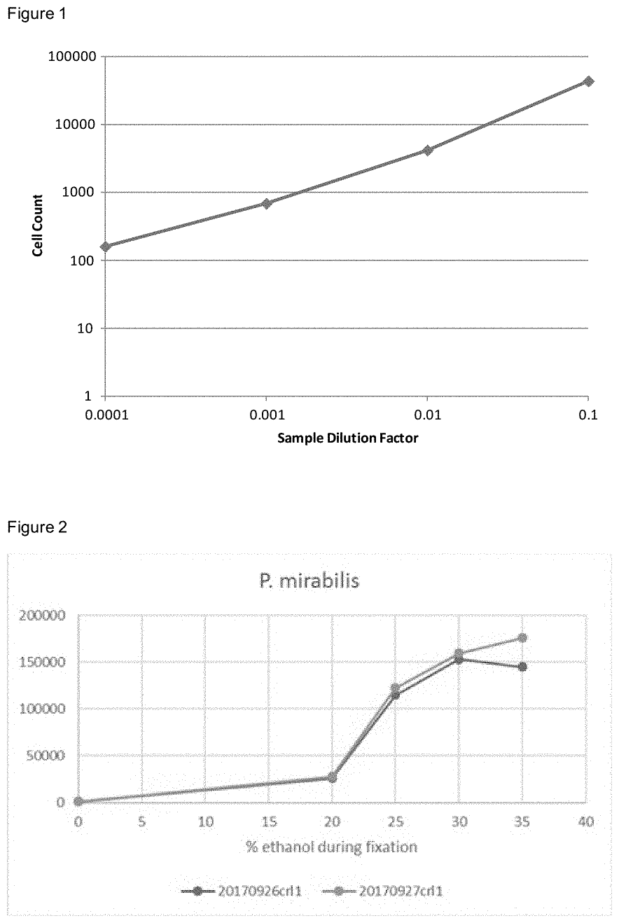

FIG. 1 shows there is a linear relationship between sample dilution and the calculated microorganism concentration using the method of the invention. Calculation of the concentration of Enterococcus faecalis is exemplified.

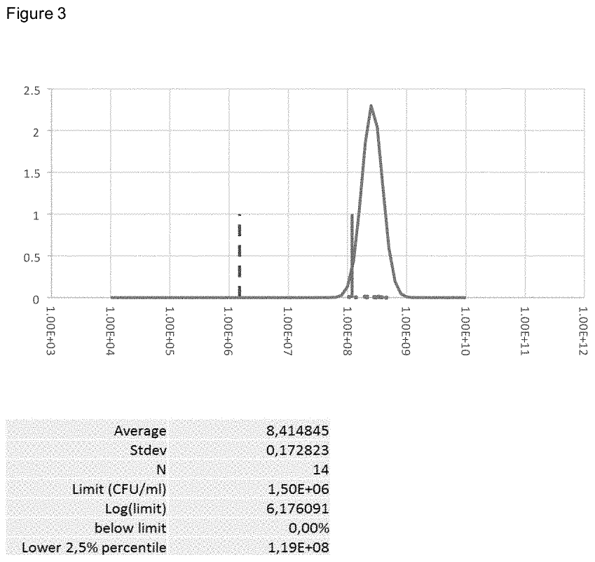

FIG. 2 shows the effect of changing the concentration of ethanol used for cell fixation in detection of microorganisms. The detection of two strains of P. mirabilis is shown (20170927cr11 is the upper line).

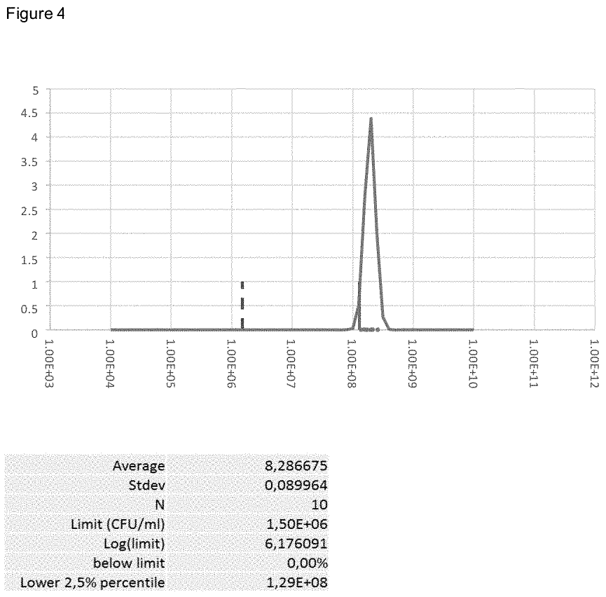

FIG. 3 shows the results of analysis of P. mirabilis as described in Example 2. The vertical dashed line is the lower limit; the vertical solid line is the lower 2.5th percentile; the normal distribution of results is also shown. The individual dots correspond to individual data points (CFU/ml as calculated by plating and colony counting). The numerical data represented is shown below the graph.

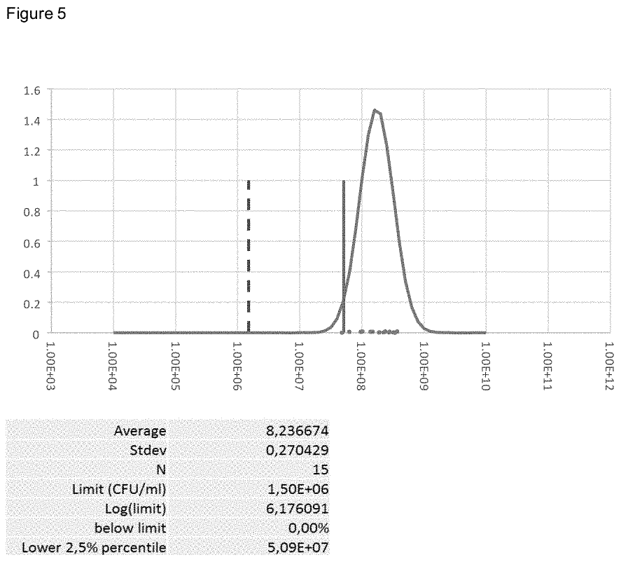

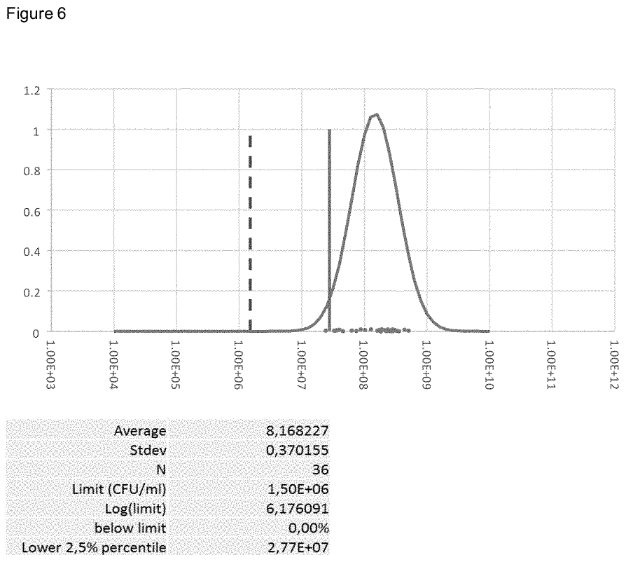

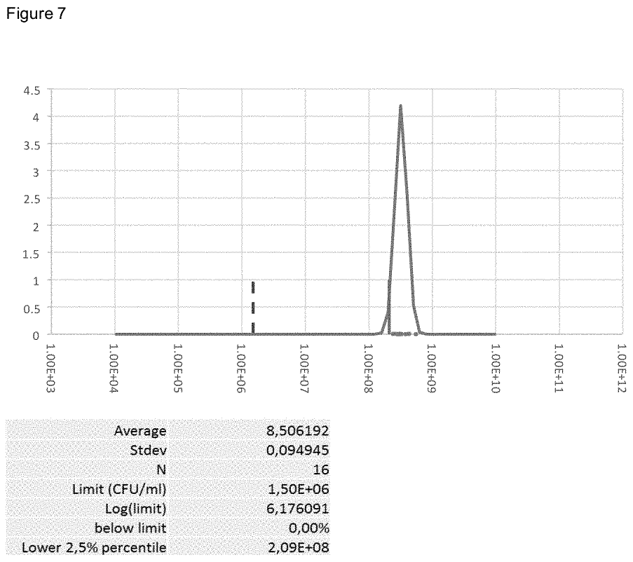

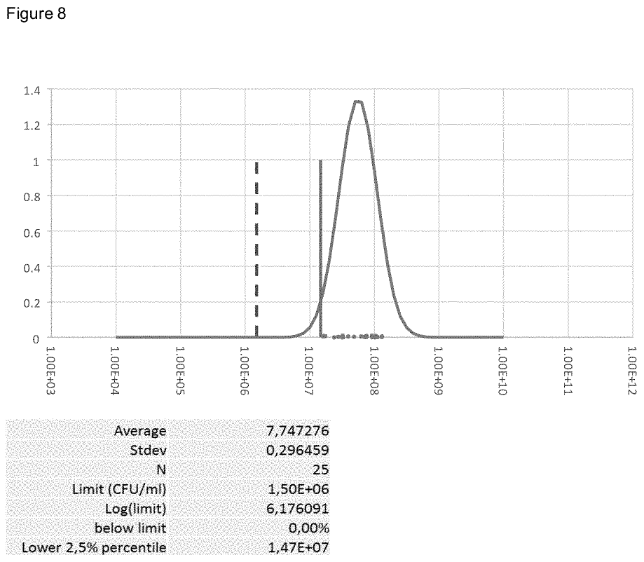

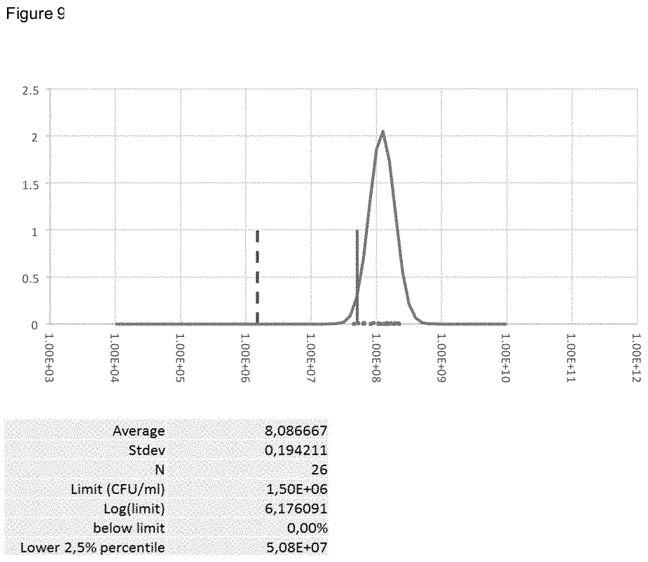

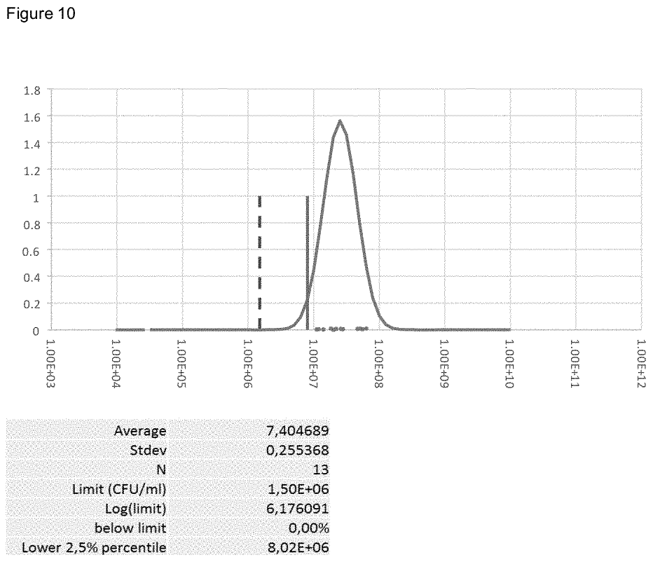

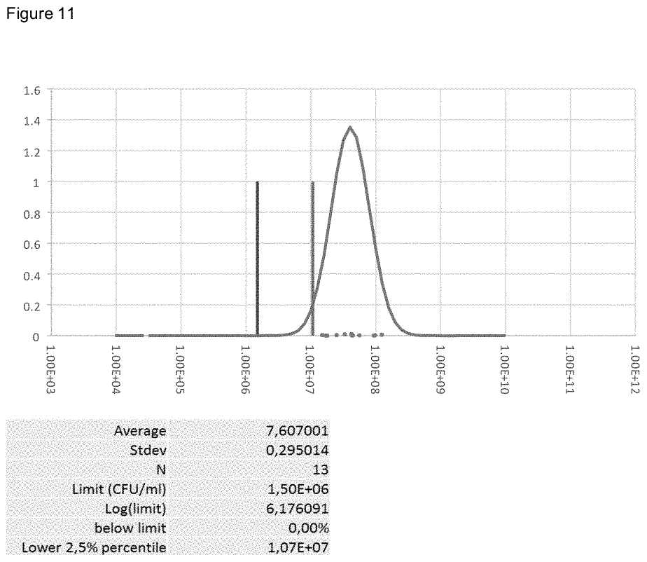

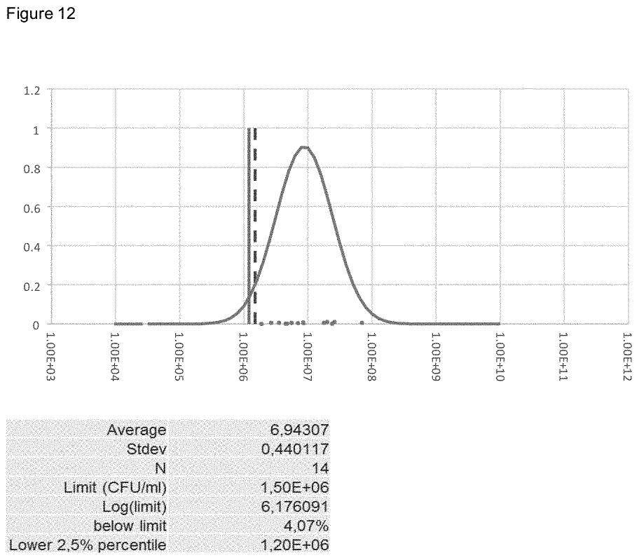

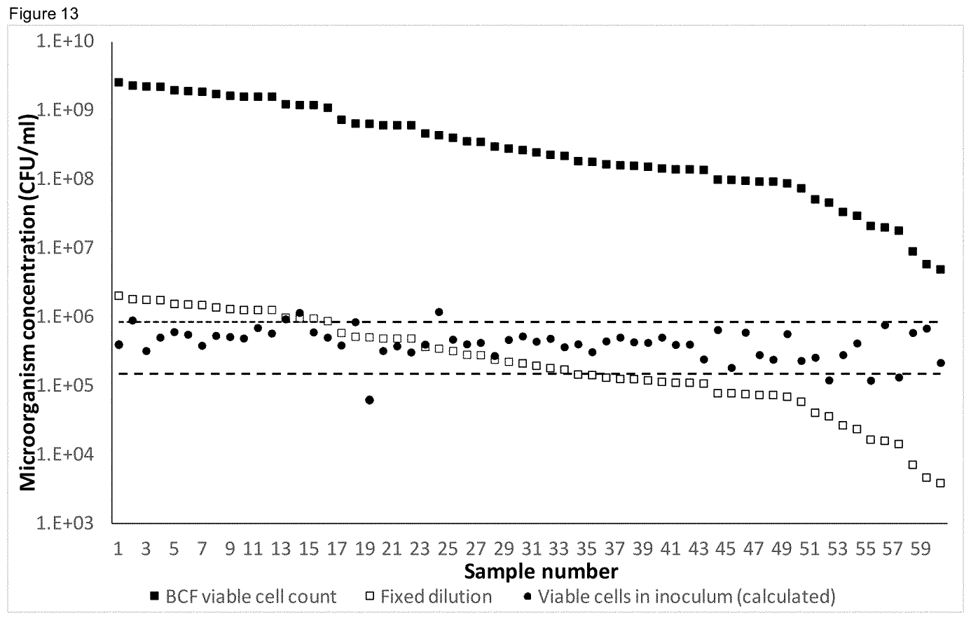

FIGS. 4-12 show the same data as FIG. 3 but for Klebsiella pneumoniae (FIG. 4), Haemophilus influenzae (FIG. 5), Escherichia coli (FIG. 6), Enterobacter cloacae (FIG. 7), Acinetobacter baumanii (FIG. 8), Streptococcus pneumoniae (FIG. 9), Pseudomonas aeruginosa (FIG. 10), Staphylococcus epidermidis (FIG. 11) and Staphylococcus aureus (FIG. 12).

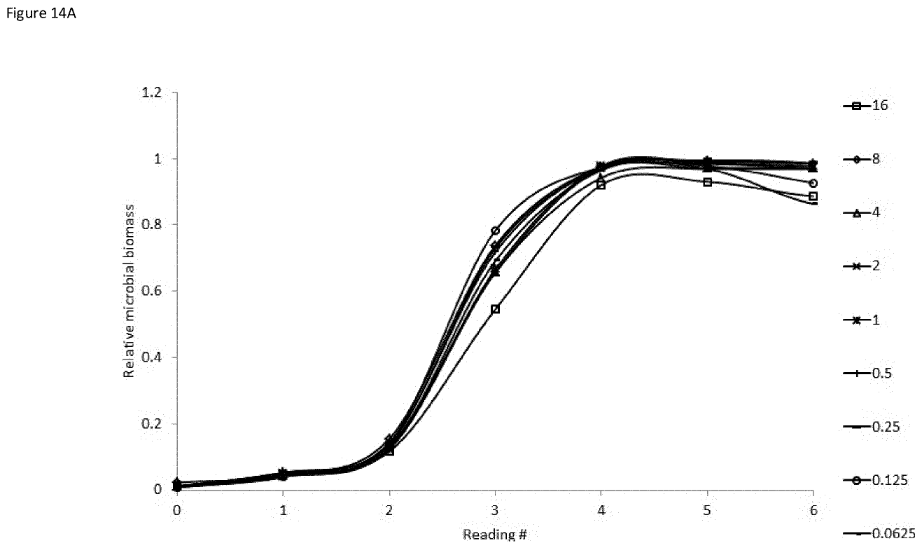

FIG. 13 shows the concentration of range of microorganism concentrations present in different positive blood culture flasks, and the resulting microorganism concentrations if a fixed dilution factor is applied to the aliquots, compared to performing steps of concentration determination and concentration adjustment. BCF sample microorganism concentration is shown as solid squares, fixed dilution sample concentration is shown as hollow squares, samples in which the concentration of microorganisms is determined/adjusted as shown as circles. Dashed lines at 5×10⁵ CFU/ml±60% are shown (EUCAST, CLSI and ICO standards).

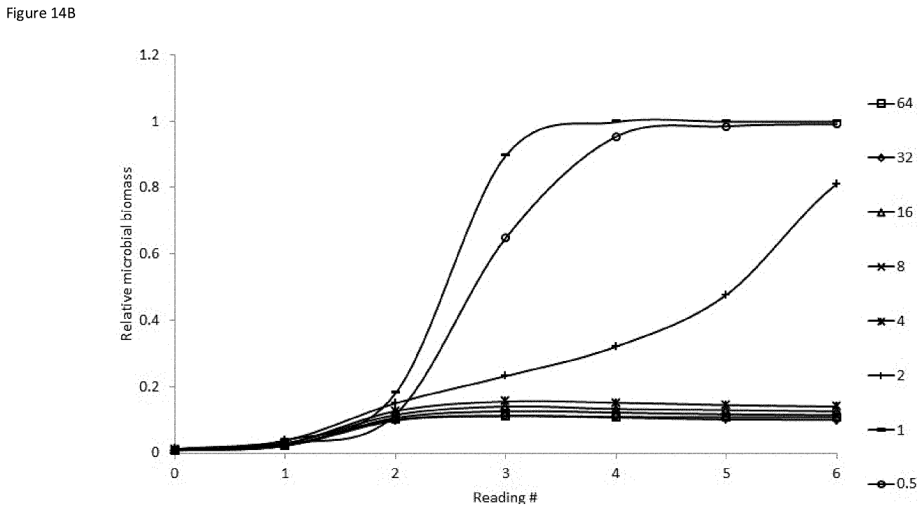

FIG. 14 shows microbial biomass over time during incubation under different conditions, for a clinical Klebsiella pneomoniae isolate. Automated microscopy images were obtained at t=30, 90, 150, 210, 270 and 330 minutes. FIG. 14A—microbial biomass in the presence of a dilution series of Trimethoprim/Sulfamethoxasole. FIG. 14B—microbial biomass in the presence of a dilution series of Piperacillin/Tazobactam. Antibiotic concentrations measured in mg/I.

### EXAMPLES

### Example 1—Microorganism Concentration Determination

Preparation of Materials

Blood Lysis Buffer:

A solution of 0.45% Brij-O10 (Sigma-Aldrich, P6136) was made in PBS (10×PBS (Sigma-Aldrich, P7059) diluted to 1× with ddH₂O), pH 7.5.

Proteinase K:

Proteinase K (Merck, 539480-1GM) was dissolved in 50 mM Tris-HCl, pH 8, to a concentration of 2.1 mg/ml, to yield a proteinase K stock solution.

SYTO BC:

5 mM SYTO BC (Thermo Fisher Scientific, S34855) was added to 1×PBS to yield a 20 μM SYTO BC stock solution.

Concentration Determination Protocol

1 ml lysis buffer was mixed with 50 μl proteinase K stock solution. The resultant lysis buffer/proteinase K mixture was added to 500 μl bacterial sample and mixed. The mixture was incubated for 7 mins at 35° C. and then filtered through a 50 mm diameter filter with 0.2 μM pore size at a rate of 4 ml/min.

Isolated bacteria were washed in 2 ml CAMBH (Thermo Fisher Scientific, T3462), and then re-suspended by back-flushing 2.5 ml CAMBH through the filter at 4 ml/min. The re-suspendate was then mixed.

20 μl re-suspendate was then mixed with from 0-20 μl 70% ethanol; the volume of the re-suspendate/ethanol mix was made up to 40 μl with ddH₂O, giving a resultant concentration of ethanol of 0-35%. The mixture was incubated for 5 mins at 35° C. 60 μl PBS was then added, and 20 μl of the resulting diluted mixture used to make 10-fold serial dilutions.

A 15 μl aliquot of each dilution sample was mixed with 15 μl SYTO BC stock solution, and the stain-sample mixtures incubated at 35° C. for 5 mins. The stain-sample mixtures were then transferred to a plate and read in an Etulama plate reader.

During reading, 50 images were obtained of microorganisms in suspension for each well, spaced 30 μm apart in the direction of the optical axis, using an emission filter at 502-561 nm to detect the SYTO BC emission peak at 509 nm. The images obtained were thresholded and subjected to analysis to determine the size, fluorescence intensity, and optionally morphology of each object corresponding to an intact microorganism to obtain an image analysis value for each aliquot. Characteristics of the microorganisms in the sample were used to select a pre-determined calibration curve for use in the concentration determination step (e.g. to determine whether the sample is a clustering or a non-clustering microorganism). One of the diluted aliquots having an image analysis value within the range of a pre-determined calibration curve was identified. The concentration of intact microorganisms in the sample was determined by comparing the image analysis value for the selected diluted aliquot with the pre-determined calibration curve.

Preparing Calibration Curves

Imaging data were collected as above for a number of different microorganisms at different, known concentrations and using different concentrations of ethanol as fixative, and the relationship between the number of objects counted and the concentration of intact microorganisms was plotted on a graph. There is a linear relationship between the number of objects counted and the concentration of intact microorganisms for the majority of microbial species when a given concentration of ethanol is used as fixative, as exemplified for Enterococcus faecalis in FIG. 1 (using 35% ethanol as fixative).

An optimal concentration of ethanol as fixative was determined for various species and strains of microorganism. For many of the tested species and strains, an optimal ethanol concentration of around 35% was identified, which enables maximal microorganism staining and hence improved detection and increased accuracy of concentration determination. An exemplary graph demonstrating concentration determination of two strains of Proteus mirabilis utilising varying concentrations of ethanol as fixative is presented in FIG. 2. For each concentration of ethanol used, the same concentration of bacteria is present in the sample. As shown an ethanol concentration of 30-35% provides optimal detection for both strains.

### Example 2—Analysis of Various Bacterial Species

Samples comprising the following species were analysed according to the method of Example 1: Proteus mirabilis (FIG. 3), Klebsiella pneumoniae (FIG. 4), Haemophilus influenzae (FIG. 5), Escherichia coli (FIG. 6), Enterobacter cloacae (FIG. 7), Acinetobacter baumanii (FIG. 8), Streptococcus pneumoniae (FIG. 9), Pseudomonas aeruginosa (FIG. 10), Staphylococcus epidermidis (FIG. 11) and Staphylococcus aureus (FIG. 12).

Between 10 and 36 samples comprising each species were analysed, as shown on the figures (see “N” value). Bacterial concentrations of each sample were determined by plating followed by CFU counting. A normal distribution was fitted to the data, and for each data set two values were marked: the lower 2.5^{th} percentile and the bacterial concentration at the lower limit of detection using the method of the invention (corresponding to 1.5×10⁶ CFU/ml).

To ensure accuracy of the method of the invention, it is preferred that at least 1 order of magnitude exists between the limit of accurate concentration determination and the concentration of the lower 2.5^{th} percentile of samples for each species. As shown in FIGS. 3-12, this is the case for all species apart from P. aeruginosa, S. epidermidis and S. aureus. For P. aeruginosa and S. epidermidis, the difference is slightly less than 1 order of magnitude; although this is not optimal, the method of the invention can nonetheless be expected to be highly accurate in measuring the concentrations of these species. For S. aureus, the limit of accurate concentration determination is at the 4th percentile. This is due to clustering of S. aureus and it is believed that separation of the S. aureus clusters, e.g. by use of a detergent, or an appropriate algorithm, will overcome this difficulty.

### Example 3—Preparation of Inocula from Positive Blood Culture Flasks

We investigated the variability in the concentration of microorganisms in positive blood culture flasks containing a variety of different Gram positive microorganisms species (Streptococcus pneumoniae, Streptococcus anginosus, Streptococcus mitis, Streptococcus pyogenes, Staphylococcus epidermidis, Staphylococcus aureus, Staphylococcus lugdunensis, Staphylococcus capitis, Staphylococcus hominis, Enterococcus faecalis, Listeria monocytogenes and Listera Grayi). Viable cell count was determined for each positive blood culture flask and a fixed dilution factor was determined based on the mean concentration of microorganisms in each blood culture flask. An aliquot from each positive blood culture flask was diluted by the fixed dilution factor.

A microbial suspension was prepared from each positive blood culture flask and viable cell count was determined for each resuspendate. The concentration of microorganisms was also determined for the resuspendate obtained from each positive blood culture flask by the method outlined above in Example 1, except that microorganisms were resuspended in 2.8 ml CAMBH. An inoculum was prepared for each sample based on the concentration of microorganisms that was determined. The actual concentration of viable cells provided in each inoculum was calculated by the following formula:

$$\frac{Resuspendate viable cell count}{Calculated viable cell count} \times 50 \text{,} 000 = Viable cells in inoculum$$

Only 28% of the positive blood culture samples diluted by the fixed dilution factor contained a concentration of viable cells which fell within the standard 5×10⁵ CFU/ml±60% for AST, whereas the inoculum prepared for 87% of the samples (adjusted based on the concentration determination method) were found to lie within this range. Results are shown in Table 1 and FIG. 13.

TABLE 1
microorganism concentrations in positive blood culture flasks and in diluted aliquots

| | | | | | | | | Viable | |
|---|---|---|---|---|---|---|---|---|---|
| | | | | | | | | cells in | |
| | | | BCF viable | Fixed | | | Calculated | inoculum | |
| | | | cell count | dilution | In range | | viable cell | (calculated) | In range |
| # | Bacteria | Strain | CFU/ml | CFU/ml | +/−60% | | count CFU/ml | CFU/ml | +/−60% |
| | | | | | | Resuspendate | | | |
| | | | | | | viable cell | | | |
| | | | | | | count CFU/ml | | | |
| 1 | E. faecalis | HV128 | 2.58E+09 | 2.04E+06 | n | 3.04E+08 | 3.80E+08 | 3.99E+05 | y |
| 2 | Strep. Pnemoniae | QM328 | 2.30E+09 | 1.82E+06 | n | 8.20E+07 | 4.60E+07 | 8.91E+05 | n |
| 3 | E. faecalis | HV400 | 2.25E+09 | 1.78E+06 | n | 1.80E+08 | 2.80E+08 | 3.21E+05 | y |
| 4 | Strep. Pnemoniae | QM145 | 2.23E+09 | 1.76E+06 | n | 1.33E+08 | 1.33E+08 | 5.00E+05 | y |
| 5 | Strep. Pnemoniae | QM329 | 1.97E+09 | 1.56E+06 | n | 1.15E+08 | 9.60E+07 | 5.99E+05 | y |
| 6 | Strep. Pnemoniae | QM145 | 1.92E+09 | 1.52E+06 | n | 8.19E+07 | 7.40E+07 | 5.53E+05 | y |
| 7 | Strep. Pnemoniae | QM327 | 1.89E+09 | 1.50E+06 | n | 4.41E+07 | 5.80E+07 | 3.80E+05 | y |
| 8 | Strep. Pnemoniae | QM330 | 1.74E+09 | 1.37E+06 | n | 7.12E+07 | 6.70E+07 | 5.32E+05 | y |
| 9 | Strep. Pnemoniae | QM328 | 1.65E+09 | 1.30E+06 | n | 1.46E+08 | 1.46E+08 | 5.10E+05 | y |
| 10 | Strep. Pnemoniae | QM327 | 1.60E+09 | 1.27E+06 | n | 7.98E+07 | 8.20E+07 | 4.87E+05 | y |
| 11 | Strep. Pnemoniae | QM329 | 1.60E+09 | 1.27E+06 | n | 1.65E+08 | 1.20E+08 | 6.88E+05 | y |
| 12 | Strep. Pnemoniae | QM330 | 1.60E+09 | 1.27E+06 | n | 1.07E+08 | 9.30E+07 | 5.75E+05 | y |
| 13 | Listeria Grayi | QM302 | 1.24E+09 | 9.81E+05 | n | 1.78E+08 | 9.60E+07 | 9.24E+05 | n |
| 14 | Listeria | HV357 | 1.21E+09 | 9.54E+05 | n | 2.74E+08 | 1.20E+08 | 1.14E+06 | n |
| | Monocyogenes | | | | | | | | |
| 15 | Strep. Pnemoniae | QM145 | 1.20E+09 | 9.49E+05 | n | 1.32E+08 | 1.10E+08 | 5.98E+05 | y |
| 16 | Strep. mitis | QM223 | 1.10E+09 | 8.70E+05 | n | 5.40E+07 | 5.40E+07 | 4.98E+05 | y |
| 17 | Strep. Mitis | QM219 | 7.38E+08 | 5.83E+05 | y | 5.66E+07 | 7.40E+07 | 3.82E+05 | y |
| 18 | E. faecalis | QM272 | 6.48E+08 | 5.13E+05 | y | 2.69E+07 | 1.60E+07 | 8.40E+05 | y |
| 19 | Strep. mitis | QM223 | 6.40E+08 | 5.06E+05 | y | 6.00E+06 | 4.80E+07 | 6.25E+04 | n |
| 20 | Staph. Aureus | HV372 | 6.12E+08 | 4.84E+05 | y | 1.21E+08 | 1.90E+08 | 3.18E+05 | y |
| 21 | Staph. Aureus | HV447 | 6.12E+08 | 4.84E+05 | y | 8.20E+07 | 1.10E+08 | 3.73E+05 | y |
| 22 | Strep. Pyogenes | QM029 | 6.11E+08 | 4.83E+05 | y | 2.08E+07 | 3.40E+07 | 3.05E+05 | y |
| 23 | Staph. Capitis | QM086 | 4.64E+08 | 3.67E+05 | y | 1.90E+07 | 2.40E+07 | 3.96E+05 | y |
| 24 | Strep. Anginosus | QM115 | 4.38E+08 | 3.46E+05 | y | 2.18E+08 | 9.20E+07 | 1.18E+06 | n |
| 25 | Strep. Pyogenes | QM149 | 4.03E+08 | 3.19E+05 | y | 5.41E+07 | 5.80E+07 | 4.66E+05 | y |
| 26 | Staph. Hominis | HV101 | 3.57E+08 | 2.82E+05 | y | 1.60E+07 | 2.00E+07 | 4.00E+05 | y |
| 27 | Staph. Epidermidis | QM370 | 3.51E+08 | 2.78E+05 | y | 3.95E+07 | 4.70E+07 | 4.20E+05 | y |
| 28 | Staph. Epidermidis | HV349 | 3.00E+08 | 2.37E+05 | y | 9.70E+06 | 1.80E+07 | 2.69E+05 | y |
| 29 | Staph. Epidermidis | HV283 | 2.80E+08 | 2.21E+05 | y | 1.20E+07 | 1.30E+07 | 4.62E+05 | y |
| 30 | Staph. Lugdunensis | QM035 | 2.68E+08 | 2.12E+05 | y | 9.23E+06 | 8.90E+06 | 5.18E+05 | y |
| 31 | Staph. Epidermidis | QM358 | 2.48E+08 | 1.96E+05 | y | 2.49E+07 | 2.50E+07 | 4.38E+05 | y |
| 32 | Staph. Epidermidis | QM358 | 2.29E+08 | 1.81E+05 | y | 2.19E+07 | 2.60E+07 | 4.79E+05 | y |
| 33 | Staph. Lugdunensis | QM036 | 2.19E+08 | 1.73E+05 | y | 8.70E+06 | 1.20E+07 | 3.62E+05 | y |
| 34 | Strep. Pyogenes | HV436 | 1.85E+08 | 1.46E+05 | n | 4.01E+07 | 5.00E+07 | 4.01E+05 | y |
| 35 | Staph. Aureus | HV011 | 1.81E+08 | 1.43E+05 | n | 2.75E+07 | 4.50E+07 | 3.06E+05 | y |
| 36 | Staph. Capitis | HV0762 | 1.65E+08 | 1.31E+05 | n | 4.66E+06 | 5.30E+06 | 4.40E+05 | y |
| 37 | Staph. Epidermidis | HV410 | 1.60E+08 | 1.27E+05 | n | 6.60E+06 | 6.60E+06 | 5.00E+05 | y |
| 38 | Staph. Epidermidis | HV410 | 1.58E+08 | 1.25E+05 | n | 1.10E+07 | 1.60E+07 | 4.28E+05 | y |
| 39 | Staph. Hominis | HV103 | 1.53E+08 | 1.21E+05 | n | 6.79E+06 | 8.10E+06 | 4.19E+05 | y |
| 40 | Strep. mitis | QM223 | 1.45E+08 | 1.14E+05 | n | 5.75E+06 | 5.75E+06 | 5.00E+05 | y |
| 41 | Staph. Epidermidis | HV410 | 1.40E+08 | 1.11E+05 | n | 1.37E+07 | 1.40E+07 | 3.93E+05 | y |
| 42 | Staph. Epidermidis | HV146 | 1.40E+08 | 1.11E+05 | n | 6.50E+06 | 8.20E+06 | 3.96E+05 | y |
| 43 | Staph. Epidermidis | HV410 | 1.37E+08 | 1.08E+05 | n | 1.06E+06 | 2.20E+06 | 2.41E+05 | y |
| 44 | Staph. Lugdunensis | HV927 | 9.91E+07 | 7.84E+04 | n | 6.21E+06 | 4.80E+06 | 6.47E+05 | y |
| 45 | Staph. Epidermidis | HV280 | 9.89E+07 | 7.82E+04 | n | 2.40E+06 | 6.60E+06 | 1.82E+05 | y |
| 46 | Strep. mitis | QM223 | 9.56E+07 | 7.56E+04 | n | 9.05E+06 | 7.70E+06 | 5.88E+05 | y |
| 47 | Staph. Capitis | HV0759 | 9.33E+07 | 7.38E+04 | n | 1.45E+07 | 2.60E+07 | 2.79E+05 | y |
| 48 | Staph. Aureus | QM062 | 9.30E+07 | 7.36E+04 | n | 2.10E+07 | 4.30E+07 | 2.39E+05 | y |
| 49 | Staph. Epidermidis | HV280 | 8.75E+07 | 6.92E+04 | n | 7.20E+06 | 6.70E+06 | 5.61E+05 | y |
| 50 | Staph. Aureus | QM216 | 7.44E+07 | 5.89E+04 | n | 1.47E+07 | 3.20E+07 | 2.29E+05 | y |
| 51 | Strep. Mitis | QM239 | 5.12E+07 | 4.05E+04 | n | 3.32E+06 | 6.50E+06 | 2.55E+05 | y |
| 52 | Staph. Aureus | HV87 | 4.59E+07 | 3.63E+04 | n | 4.74E+06 | 2.00E+07 | 1.19E+05 | n |
| 53 | Strep. Anginosus | HV018 | 3.38E+07 | 2.67E+04 | n | 1.39E+06 | 2.50E+06 | 2.78E+05 | y |
| 54 | Staph. Hominis | HV414 | 2.97E+07 | 2.35E+04 | n | 7.24E+06 | 8.80E+06 | 4.11E+05 | y |
| 55 | Staph. Epidermidis | HV280 | 2.10E+07 | 1.66E+04 | n | 2.00E+06 | 8.50E+06 | 1.18E+05 | n |
| 56 | Strep. Anginosus | HV018 | 2.01E+07 | 1.59E+04 | n | 1.98E+06 | 1.30E+06 | 7.63E+05 | y |
| 57 | Staph. Aureus | QM079 | 1.80E+07 | 1.42E+04 | n | 3.70E+06 | 1.40E+07 | 1.32E+05 | n |
| 58 | Strep. mitis | QM134 | 8.99E+06 | 7.11E+03 | n | no colonies | 2.00E+07 | 5.83E+05 | y |
| 59 | Strep. mitis | QM215 | 5.87E+06 | 4.64E+03 | n | 8.90E+05 | 6.60E+05 | 6.74E+05 | y |
| 60 | Staph. Aureus | QM013 | 4.90E+06 | 3.88E+03 | n | 3.80E+06 | 8.90E+06 | 2.13E+05 | y |
| | | | | | | Resuspendate | | | |
| | | | | | | viable count | | | |
| | | | | | | CFU/ml | | | |
| | Mean BCF CFU/ml | | 6.32E+08 | 500000 | 60 | 3.04E+08 | | 4.60E+05 | 60 |
| | S.D. | | 7.39E+08 | | 17 | 8.20E+07 | | 2.24E+05 | 52 |
| | | | | | 28% | 1.80E+08 | | | 87% |

| | | |
|---|---|---|
| Calc. Dilution factor | 1264 | 1.33E+08 |
| to inoculum | | |

### Example 4—Isolation, Concentration Determination and Antibiotic Susceptibility of a Spiked Positive Blood Culture Flask Using a Clinical Isolate of Klebsiella pneumoniae

Preparation of Materials

Preparation of Positive Blood Culture Sample

Clinical isolates grown on agar plate were individually suspended in PBS and adjusted to 0.5 McFarland. A 1:100 dilution of this was added together with 9 ml of blood from a healthy donor to a blood culture flask (BD Bactec Plus Aerob) and incubated in a blood culture cabinet overnight. In the morning, after the BCF had turned positive a 500 μl aliquot of the positive BCF were used for the subsequent analysis.

Sample Preparation:

500 μl of the positive BCF were added to a consumable allowing automated sample preparation and concentration adjustment and the concentration of microorganisms was determined in an automated system, implementing the method as described in Example 1, except that microorganisms were resuspended in 2.8 ml CAMBH. The operation of such a system using the consumable is described in more detail in our co-pending application GB 1806505.2. The value of the concentration determination was compared to a pre-determined standard curve and the concentration of microorganisms in the recovered suspension was automatically adjusted to the desired concentration (5×10\hat{ }5 CFU/ml). For this experiment an aliquot of the concentration adjusted bacteria were plated on an agar plate to determine viable cell count to provide a control measure for the process.

$$\begin{matrix} & Desired CFU \text{/} & Actual CFU \text{/} \\ \underline{Isolate} & \underline{ml after conc adjust} & \underline{ml after conc adjust} \\ Klebsiella pneumonia & 5 \text{,} 0 E + 05 & 3 \text{,} 5 E + 05\end{matrix}$$

AST

The concentration adjusted sample in CAMBH was added using an automated pipette via central access ports to a 336-well AST-disc pre-filled with dried antibiotics in various concentrations.

Each well contain 20 μl of sample and is incubated at 35° C. An initial reading was taken after 30 minutes (reading 0). Subsequent readings (readings 1-6) were taken every hour up to 5.5 hours total AST time by imaging by automated microscopy. The automated microscope as used is described in more detail in our co-pending application PCT/EP2018/085692.

MIC Calling

Images were analysed by converting the image into a biomass value and MIC called, as described in WO 2017/216312. Microbial biomass at each time point under different concentrations of antibiotics (mg/I) were determined for different antibiotics, e.g. as shown in FIG. 14A (Trimethoprim/Sulfamethoxazole) and FIG. 14B (Piperacillin/Tazobactam).

Results

MIC antibiotic concentrations (measured in mg/I) were determined for a range of antibiotics. Each antibiotic were present in triplicates in the AST-consumable in this experiment. Results are shown in Table 2.

TABLE 2
MIC values

| | #1 | #2 | #3 |
|---|---|---|---|
| Amoxicillin/ | 8 | 16 | 16 |
| clavulanic acid | | | |
| Amikacin | 1 | 2 | 1 |
| Aztreonam | 32 | 32 | 32 |
| Ceftazidime | <=0.125 | <=0.125 | <=0.125 |
| ceftazidime-avibactam | 16 | 32 | 32 |
| Cefepime | 8 | 8 | 8 |
| Ciprofloxacin | 2 | 4 | 4 |
| Colistin | 0.5 | 1 | 1 |
| Cefoxitine | 2 | 4 | 2 |
| Ceftriaxone | >8 | >8 | >8 |
| Ceftolozane-tazobactam | 0.25 | 0.25 | 0.25 |
| Cefotaxime | >8 | >8 | >8 |
| Ertapenem | <=0.015625 | 0.03125 | <=0.015625 |
| Gentamicin | 0.5 | 0.5 | 0.5 |
| Imipinem | <=0.5 | <=0.5 | <=0.5 |
| Levofloxacin | 0.5 | 0.5 | 0.5 |
| Meropenem | <=0.0625 | <=0.0625 | <=0.0625 |
| Piperacillin/Tazobactam | 4 | 4 | 4 |
| Tigecyclin | 0.5 | 0.5 | 0.5 |
| Tobramycin | 2 | 2 | 2 |
| Trimethoprim/ | >16 | >16 | >16 |
| Sulfamethoxasole | | | |

1. A method of preparing a suspension of intact microorganisms from a sample containing microorganisms and mammalian cells, said method comprising:
a. providing a sample containing microorganisms and mammalian cells;
b. contacting said sample with a buffer solution, a detergent and one or more proteases, wherein said buffer solution has a pH of at least pH 6 and less than pH 9 to allow lysis of mammalian cells present in said sample;
c. filtering the mixture obtained in step (b) through a filter suitable for retaining microorganisms, wherein said filtering removes the lysed mammalian cells from the mixture;
d. recovering the microorganisms retained by the filter in step (c), wherein said recovery comprises resuspending the microorganisms in a liquid to provide a suspension comprising the recovered intact microorganisms; and
e. determining the concentration of microorganisms in said suspension, wherein the concentration of microorganisms is determined by a method comprising:
i. contacting an aliquot of said suspension with an alcohol and/or heating an aliquot of said suspension;
ii. optionally diluting one or more aliquots of said suspension to provide one or more diluted aliquots at one or more dilution values, wherein said dilution takes place before, during and/or after step (i);
iii. contacting at least a portion of an aliquot of step (e)(i) or (e)(ii) with a single fluorescent stain capable of binding to DNA to provide a suspension-stain mixture, wherein said stain has an emission wavelength;
iv. imaging the suspension-stain mixture of step (e)(iii) at the emission wavelength of the fluorescent stain and determining an image analysis value for the number of objects corresponding to microorganisms in the imaged mixture; and
v. comparing an image analysis value obtained in step (e)(iv) for a said aliquot of step (e)(iii) to a pre-determined calibration curve, thereby to determine the concentration of microorganisms in the suspension.

2. The method of claim 1, wherein said method further comprises, as step (f), adjusting the concentration of microorganisms in at least a portion of said suspension.

3. The method of claim 2, wherein step (f) comprises adjusting the concentration of microorganisms in at least a portion of the suspension before the concentration of microorganisms in the suspension is determined.

4. The method of claim 2 or 3, wherein step (f) comprises adjusting the concentration of microorganisms in at least a portion of the suspension after the concentration has been determined in step (e).

5. The method of any one of claims 2 to 4, wherein the concentration is adjusted by dilution.

6. The method of any one of claims 1 to 5, wherein the stain of step (e)(iii) is a cell-permeable stain.

7. The method of any one of claims 1 to 6, wherein the sample is a clinical or veterinary sample or a culture of a clinical or veterinary sample.

8. The method of any one of claims 1 to 7, wherein the buffer solution has a pH of 6.5 to 8.5, or a pH of 6.5 to 8 or 7 to 8.

9. The method of any one of claims 1 to 8, wherein the buffer solution has a pH of 7.5.

10. The method of any one of claims 1 to 9, wherein the detergent is a non-ionic detergent.

11. The method of claim 10 wherein the non-ionic detergent is Brij-O10.

12. The method of any one of claims 1 to 11, wherein the concentration of the detergent is between 0.1 and 5% w/v, or between 0.1 and 1% w/v.

13. The method of claim 12, wherein the concentration of the detergent is 0.45% w/v.

14. The method of any one of claims 1 to 13, wherein the protease is Proteinase K.

15. The method of any one of claims 1 to 14, wherein step (b) comprises contacting the sample with a composition comprising (i) a lysis buffer comprising PBS pH 7.5, 0.45% w/v Brij-O10, and (ii) Proteinase K.

16. The method of any one of claims 1 to 15, wherein filtration step (c) comprises filtering the mixture using a filter having a pore size of less than 0.5 μm, preferably less than 0.25 μm.

17. The method of any one of claims 1 to 16, wherein recovering microorganisms from the filter comprises back-flushing liquid through the filter.

18. The method of any one of claims 1 to 17, wherein in step (d) the microbial cells are resuspended in a liquid growth medium suitable for culturing microorganisms.

19. The method of any one of claims 1 to 18, wherein the filter is washed between steps (c) and (d).

20. The method of any one of claims 1 to 19, wherein the alcohol of step (e)(i) is ethanol.

21. The method of claim 20, wherein in step (e)(i) ethanol is added to said suspension to a resultant concentration of 30 to 40% v/v, preferably 35% (v/v).

22. The method of any one of claims 1 to 21, wherein in step (e)(ii) the suspension is diluted with a buffer.

23. The method of claim 22, wherein the buffer is PBS.

24. The method of any one of claims 1 to 23, wherein the fluorescence intensity of said fluorescent stain at said emission wavelength is enhanced when the stain is bound to nucleic acid.

25. The method of claim 24, wherein said fluorescent stain is an unsymmetrical cyanine dye.

26. The method of any one of claims 1 to 25, wherein said fluorescent stain has excitation and emission wavelengths in the wavelength range 350-700 nm.

27. The method of claim 26, wherein said fluorescent stain is a green-fluorescent stain.

28. The method of claim 24 or 27, wherein the fluorescent stain is a SYTO stain.

29. The method of claim 28, wherein the SYTO stain is SYTO BC.

30. The method of any one of claims 1 to 29, wherein said method comprises diluting aliquots of said suspension to provide two or more diluted aliquots at different dilution values, wherein said two or more aliquots are prepared simultaneously during step (e)(ii), or sequentially wherein a second or further diluted aliquot is prepared after step (e)(iv) and/or (e)(v).

31. The method of claim 30, wherein steps (e)(iii) and (e)(iv) are performed on two or more aliquots at different dilution values, and wherein step (e)(v) comprises identifying an aliquot which comprises an image analysis value within the range of a pre-determined calibration curve, and comparing the image analysis value for said aliquot to said pre-determined calibration curve, thereby to determine the concentration of intact microorganisms in said suspension.

32. The method of claim 31, wherein steps (e)(iii) and (e)(iv) are performed on each aliquot simultaneously.

33. The method of claim 31, wherein steps (e)(iii) and (e)(iv) are performed on each aliquot sequentially.

34. The method of any one of claims 1 to 33, wherein an image is obtained at one or more focal planes through the suspension-stain mixture.

35. The method of claim 34, wherein said imaging comprises obtaining a series of 2-D images along an optical axis, wherein each image is obtained at a different position along the optical axis through a volume of the suspension-stain mixture.

36. The method of any one of claims 1 to 35, wherein step (e)(iii) of contacting with the stain is performed at a temperature of greater than 4° C.

37. The method of any one of claims 1 to 36, wherein in the contacting of step (e)(iii) the aliquot or diluted aliquot, or portion thereof, is incubated with the stain for a time period of 1 to 20 minutes.

38. The method of any one of claims 1 to 37, wherein the imaging in step (e)(iv) is carried out at room temperature.

39. The method of any one of claims 1 to 38, wherein in the imaging step (e)(iv) it is identified whether the microorganisms are clustering or non-clustering and a calibration curve is used which is predetermined for clustering or non-clustering microorganisms.

40. The method of any one of claims 1 to 39, wherein the images are analysed for fluorescence intensity and/or size of each enumerated object, and optionally morphology of each enumerated object.

41. The method of any one of claims 1 to 40, wherein the images are analysed for maximum fluorescence intensity, median fluorescence intensity and/or area of each enumerated object.

42. The method of any one of claims 1 to 41, wherein the images are analysed for maximum, median and/or mean fluorescence intensity and/or area of the population of objects.

43. A method for determining the antimicrobial susceptibility of a microorganism in a sample, said method comprising:
(i) providing a sample containing a viable microorganism and mammalian cells;
(ii) performing steps (b)-(d) as defined in any one of claims 1 to 40 on said sample, to yield a suspension of the viable microorganisms;
(iii) performing step (e) as defined in any one of claims 1 to 40 to determine the concentration of microbial cells in the suspension;
(iv) inoculating a series of test microbial cultures for an antibiotic susceptibility test (AST) using the suspension of step (ii), wherein the series of test microbial cultures comprises at least two different growth conditions, wherein the different growth conditions comprise one or more different antimicrobial agents, and each antimicrobial agent is tested at two or more different concentrations; and
(v) assessing the degree of microbial growth in each growth condition;
wherein the concentration of microbial cells in said suspension or said test microbial cultures is adjusted if necessary to a desired or pre-determined concentration; and
wherein the degree of microbial growth in each growth condition is used to determine at least one value indicative of the susceptibility of the microorganism in the sample to at least one antimicrobial agent.

44. The method of claim 43, wherein at least one MIC and/or SIR value is determined to determine the antimicrobial susceptibility of said microorganism in said sample.

45. The method of claim 43 or claim 44, wherein, based on the concentration determined in step (iii), the concentration of at least a portion of the suspension of step (ii) is adjusted to provide an inoculum for inoculating the test microbial cultures in step (iv).

46. The method of any one of claims 43 to 45, wherein the step of adjusting the concentration comprises a dilution based on the concentration determined in step (iii).

47. The method of claim 46, wherein following step (iii), at least a portion of the suspension of step (ii) is diluted to provide an inoculum for step (iv).

48. The method of any one of claims 43 to 47, wherein the concentration of microorganisms in the inoculated microbial test cultures is in the range 4.5×10⁵±80% or 5×10⁵±60%.

49. The method of any one of claims 43 to 48, wherein at least one of the test microbial cultures comprises fastidious medium.

50. The method of any one of claims 43 to 45 or 48 to 49, wherein the concentration adjustment comprises culturing or further culturing the suspension.

51. The method of any one of claims 43 to 50, wherein if the concentration of microorganisms in the suspension is below 1×10⁶ microorganisms, the AST assay is not performed with the suspension.
